(19) United States
(12) Patent Application Publication
(10) Pub. No.: US 2026/0124245 A1
(43) Pub. Date: May 7, 2026

(54) METHODS AND COMPOSITIONS FOR USING LEUCINE ZIPPERS FOR CROSSLINKING OF CELLS AND DRUG CARRIERS

(71) Applicant: The University of North Carolina at Chapel Hill, Chapel Hill, NC (US)

(72) Inventors: Juliane Nguyen, Chapel Hill, NC (US); Natalie Jasiewicz, Chapel Hill, NC (US)

(21) Appl. No.: 18/997,668

(22) PCT Filed: Jul. 28, 2023
(86) PCT No.: PCT/US2023/071186

§ 371 Date: Jan. 22, 2025

Related U.S. Application Data
(60) Provisional application No. 63/393,417, filed on Jul. 29, 2022.

(51) Int. Cl. A61K 35/28; A61K 38/16; A61P 9/10; C07K 14/00

(52) U.S. Cl. CPC A61K 35/28; A61K 38/16; A61P 9/10; C07K 14/001

(57) ABSTRACT

Non-invasive, in situ forming depots for delivery of a therapeutic agents, containing heterodimerizing, synthetic leucine zippers for physical crosslinking mediated by competition-based dimerization. The heterodimerizing, synthetic leucine zippers form a self-assembling depot of the therapeutic agent at a target site in vivo. A library of such heterodimerizing, synthetic leucine zippers is provided, as well as methods of treating subjects using the same.

### PRIORITY CLAIM

The present application claims the benefit of U.S. Provisional Patent Application Ser. No. 63/393,417, filed Jul. 29, 2022, the disclosure of which is incorporated herein by reference in its entirety.

### GRANT STATEMENT

This invention was made with government support under Grant Number EB023262 and HL161456 awarded by the National Institutes of Health. The government has certain rights in the invention.

### TECHNICAL FIELD

The subject matter disclosed herein relates generally to methods and compositions for using leucine zippers for crosslinking of cells and drug carriers. More particularly, the subject matter disclosed herein relates to compositions, formulations and methods for treating a condition using a leucine zipper therapeutic.

### REFERENCE TO SEQUENCE LISTING XML SUBMITTED ELECTRONICALLY

The content of the Sequence Listing XML filed using Patent Center as an XML file (Name: 4210_0525WO 7-28-23-12.xml; Size: 16.992 bytes; and Date of Creation: Jul. 28, 2023) is incorporated herein by reference in its entirety.

### BACKGROUND

Myocardial infarction remains a leading cause of death worldwide. Although the current standard of care for patients focuses on restoring perfusion as quickly as possible with percutaneous coronary interventions, patients are often left with dysregulated endogenous infarct repair and chronic severe morbidities, including ventricular arrhythmias and heart failure due to insufficient cardiac contractile function. The drug delivery and regenerative medicine fields have focused on cell delivery, particularly mesenchymal stem cell delivery, to combat these downstream effects due to their regenerative abilities. Despite these efficacious tools, delivery to the target site after intravenous injection remains highly ineffective, and traditional cell and other therapeutic interventions often require invasive intramyocardial injection or implantation. These invasive injections or implantations come with a variety of mechanical and biological risks such as arrhythmia, induction of fibrosis, and elevated serum myocardial biomarkers, indicative of cardiomyocyte death. Even with local delivery, promising in vitro MSC therapies, extracellular vesicle (EV) therapies, and other therapies are often plagued by poor therapeutic accumulation and retention at the infarct site of interest. Rapid cellular clearing diminishes effective doses and therefore potential therapeutic efficacy.

Thus, there remains an urgent need for non-invasive approaches that enhance the accumulation and retention of cell-based therapeutics, drug carriers, delivery vehicles, nanocarriers, therapeutics, biologics, nucleic acids, etc. at the disease site. To address this gap, efforts were undertaken to develop a novel delivery strategy to enhance the accumulation and retention of cells in the injured myocardium. The goal is to create a modular platform of cells, drug carriers, delivery vehicles, nanocarriers, therapeutics, biologics, nucleic acids, etc. that crosslink to a scaffold at the infarct site or in the injured myocardium to serve as a drug depot. In some embodiments, the design criteria for the scaffold include, but are not limited to, (1) enhanced accumulation at the infarct site/injured myocardium, (2) prolonged retention, and/or (3) in case of drug complications or immunogenic reactions, the possibility to dissolve the scaffold into its individual subunits for elimination.

### SUMMARY

This summary lists several embodiments of the presently disclosed subject matter, and in many cases lists variations and permutations of these embodiments. This summary is merely an example of the numerous and varied embodiments. Mention of one or more representative features of a given embodiment is likewise for purposes of example. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features.

In one aspect, a non-invasive, in situ forming depot for delivery of a therapeutic agent, includes one or more heterodimerizing, synthetic leucine zippers for physical crosslinking mediated by competition-based dimerization, and a therapeutic agent, where the one or more heterodimerizing, synthetic leucine zippers form a self-assembling depot of the therapeutic agent at a target site in vivo. The non-invasive, in situ forming depot for delivery of a therapeutic agent may also include where the heterodimerizing, synthetic leucine zippers amplify an available binding area at a target site in vivo in a layer-by-layer fashion. In some aspects, the therapeutic agent includes an affinity or attraction to a target site in vivo, optionally includes a targeting ligand on a surface of the therapeutic agent, to enhance accumulation of the therapeutic agent at the target site. In some aspects, the non-invasive, in situ forming depots for delivery of a therapeutic agent achieve significantly enhanced retention and accumulation of the therapeutic agent at the target site. In some aspects, the therapeutic agent includes a cell, optionally a stem cell, a vesicle, an extracellular vesicle (EV), a nanoparticle, a microparticle, a small molecule, a biotherapeutic, a biologic, a pharmaceutical, and combinations thereof. In some aspects, the one or more heterodimerizing, synthetic leucine zippers are attached to a surface of the therapeutic agent, optionally attached via crosslinking, physical attachment or genetic fusion, optionally a heterobifunctional crosslinker. In some aspects, the therapeutic agent includes a stem cell, where the stem cell includes a targeting ligand on a surface of the stem cell. In some aspects, the therapeutic agent includes a mesenchymal stem cell with a natural ability to migrate to sites of inflammation in vivo.

In some aspects, the non-invasive, in situ forming depots are configured to be sequentially administered to a subject, whereby one or more layers of the in situ forming depots can be formed at a target site. In some aspects, the non-invasive, in situ forming depots are configured to be sequentially administered to a subject, whereby each sequential administration is configured to provide a different therapeutic agent and/or a different dosage of the therapeutic agent. In some aspects, the non-invasive, in situ forming depots are configured for a layer-by-layer dosing strategy. In some aspects, the non-invasive, in situ forming depots are configured for intravenous administration to a subject, optionally for subcutaneous, intramuscular, local, intracranial and/or intraarterial administration. In some aspects, the multivalent crosslinking of the one or more leucine zippers increases resistance to venous washout, allowing the therapeutic agent to persist at the target site long term. In some aspects, the non-invasive, in situ forming depots, including the therapeutic agent, are configured to persist at the target site for at least about 5 days, about 10 days, about 15 days, about 20 days, about 30 days, about 50 days, about 100 days, or more. In some aspects, the non-invasive, in situ forming depots, including the therapeutic agent, are configured to deposit at the target site at a concentration of at least about 2 times, about 5 times, about 10 times, about 20 times, about 50 times, or more, as compared to a therapeutic agent without the non-invasive, in situ forming depots.

In some aspects, the one or more leucine zippers comprise an amino acid or nucleotide sequence of any of SEQ ID NOs. 1-8, or a variant thereof substantially identical to any of SEQ ID NOs. 1-8, optionally where the variant has a homology of at least about 75%, 80%, 85%, 90%, 95%, 99% or more as compared to any of SEQ ID NOs. 1-8. In some aspects, the one or more leucine zippers comprise an amino acid sequence of SEQ ID NOs. 1, 3, 5 or 7, or a variant thereof, where amino acids 22-47 of SEQ ID NOs. 1, 3, 5 or 7 is substantially conserved. In some aspects, the one or more leucine zippers comprise one or more mutated amino acids with increased hydrophobic content. In some aspects, the one or more leucine zippers comprise one or more hydrophilic non-interacting surfaces and one or more hydrophobic interfacial contacts. In some aspects, the non-invasive, in situ forming depots are configured to be disassembled via competition-mediated disassembly.

Provided in some embodiments is a method of treating, ameliorating and/or preventing a condition in a subject, the method includes providing a subject in need of treatment, amelioration and/or prevention of a condition, and administering to the subject a non-invasive, in situ forming depot as disclosed herein. In some aspects, the target site is an infarct site, where the therapeutic agent includes a stem cell. In some aspects, the stem cell maintains a regenerative wound healing property and/or capacity to differentiate after deposition at the target site.

In one aspect of the methods, a leucine zipper includes an amino acid sequence of any of SEQ ID NOs. 1, 3, 5 or 7, or a substantially identical variant thereof, optionally where the variant has a homology of at least about 75%, 80%, 85%, 90%, 95%, 99% or more as compared to any of SEQ ID NOs. 1, 3, 5 or 7, optionally where amino acids 22-47 of SEQ ID NOs. 1, 3, 5 or 7 are substantially conserved. The leucine zipper may also include where the leucine zipper includes an amino acid sequence of SEQ ID NO. 1, or a substantially identical variant thereof, optionally where the variant has a homology of at least about 75%, 80%, 85%, 90%, 95%, 99% or more as compared to SEQ ID NO. 1, optionally where amino acids 22-47 of SEQ ID NO. 1 are substantially conserved. The leucine zipper may also include where the leucine zipper includes an amino acid sequence of SEQ ID NO. 3, or a substantially identical variant thereof, optionally where the variant has a homology of at least about 75%, 80%, 85%, 90%, 95%, 99% or more as compared to SEQ ID NO. 3, optionally where amino acids 22-47 of SEQ ID NO. 3 are substantially conserved. The leucine zipper may also include where the leucine zipper includes an amino acid sequence of SEQ ID NO. 5, or a substantially identical variant thereof, optionally where the variant has a homology of at least about 75%, 80%, 85%, 90%, 95%, 99% or more as compared to SEQ ID NO. 5, optionally where amino acids 22-47 of SEQ ID NO. 5 are substantially conserved. The leucine zipper may also include where the leucine zipper includes an amino acid sequence of SEQ ID NO. 7, or a substantially identical variant thereof, optionally where the variant has a homology of at least about 75%, 80%, 85%, 90%, 95%, 99% or more as compared to SEQ ID NO. 7, optionally where amino acids 22-47 of SEQ ID NO. 7 are substantially conserved.

Provided in some embodiments, a pharmaceutical composition includes a leucine zipper-based therapeutic in combination with a stem cell therapeutic, where one or more stem cells in the stem cell therapeutic are tagged with a leucine zipper as disclosed herein. In one aspect, a leucine zipper includes nucleotide sequence of any of SEQ ID NOs. 2, 4, 6 or 8, or a substantially identical variant thereof, optionally where the variant has a homology of at least about 75%, 80%, 85%, 90%, 95%, 99% or more as compared to any of SEQ ID NOs. 2, 4, 6 or 8. The leucine zipper may also include where the leucine zipper includes a nucleotide sequence of SEQ ID NO. 2, or a substantially identical variant thereof, optionally where the variant has a homology of at least about 75%, 80%, 85%, 90%, 95%, 99% or more as compared to SEQ ID NO. 2. The leucine zipper may also include where the leucine zipper includes a nucleotide sequence of SEQ ID NO. 4, or a substantially identical variant thereof, optionally where the variant has a homology of at least about 75%, 80%, 85%, 90%, 95%, 99% or more as compared to SEQ ID NO. 4. The leucine zipper may also include where the leucine zipper includes a nucleotide sequence of SEQ ID NO. 6, or a substantially identical variant thereof, optionally where the variant has a homology of at least about 75%, 80%, 85%, 90%, 95%, 99% or more as compared to SEQ ID NO. 6. The leucine zipper may also include where the leucine zipper includes a nucleotide sequence of SEQ ID NO. 8, or a substantially identical variant thereof, optionally where the variant has a homology of at least about 75%, 80%, 85%, 90%, 95%, 99% or more as compared to SEQ ID NO. 8.

The non-invasive, in situ forming depot for delivery of a therapeutic agent may also include that the one or more heterodimerizing, synthetic leucine zippers are attached to a surface of an EV via crosslinking, physical attachment or genetic fusion. The non-invasive, in situ forming depot for delivery of a therapeutic agent may also include where the one or more heterodimerizing, synthetic leucine zippers cause physical multivalent crosslinking, where physical multivalent crosslinking causes retention and accumulation of the therapeutic agent at the target site. The non-invasive, in situ forming depot for delivery of a therapeutic agent may also include where each layer serves as an additional capturing surface for a subsequent dose of a non-invasive, in situ forming depot.

The method may also include where the subject is a human subject, optionally where the subject is suffering from an inflammatory condition, optionally where the subject is suffering from myocardial infarction, ischemia, cancer, arthritis, joint disease, and/or limb ischemia. The method may also include where the subject is suffering from myocardial infarction or is susceptible to suffering from myocardial infarction. The method may also include where the subject is administered sequential doses of the non-invasive, in situ forming depot, optionally where the therapeutic agent and/or dosage varies between the sequential doses. The method may also include where the subject administered the non-invasive, in situ forming depot show improved cardiac function, defined as higher fractional shortening (FS), optionally about 10% to about 130% higher, decreased systolic LV diameter), optionally about 10% to about 40% lower, improved ejection fraction), optionally about 10% to about 100% higher, and/or less fibrosis), optionally about 15% to about 60% lower. The method of any may also include where the subject administered the non-invasive, in situ forming depot retains the therapeutic agent at the depot site for at least about 5, 6, 7, 8, 9, 10, 11, 12, 13, 14, 15, 16, 17, 18, 19, 20, 21, 22, 23, 24 or 25 days.

The pharmaceutical composition may also include where the leucine zipper-based therapeutic includes a leucine zipper selected from a library of heterodimerizing, synthetic leucine zippers for physical crosslinking mediated by competition-based dimerization. The pharmaceutical compositions may also include where the pharmaceutical composition is configured for non-invasive, targeted, systemic administration in a subject. The pharmaceutical compositions may also include where the pharmaceutical composition provides stem cell as mediators of wound healing, optionally where the stem cells are configured to self-renew, immunomodulate, and/or differentiate into a variety of cell types for regenerative medicine. The pharmaceutical composition may also include wherein the stem cell therapeutic comprises an extracellular vesicle (EV).

Although some of the aspects of the subject matter disclosed herein have been stated hereinabove, and which are achieved in whole or in part by the presently disclosed subject matter, other aspects will become evident as the description proceeds when taken in connection with the accompanying drawings as best described herein below.

### BRIEF DESCRIPTION OF THE DRAWINGS

The features and advantages of the present subject matter will be more readily understood from the following detailed description which should be read in conjunction with the accompanying drawings that are given merely by way of explanatory and non-limiting example, and in which:

FIG. 1 depicts a schematic representation of layer-by-layer in situ cellular depot formation. Cells are initially decorated with either a Base Leucine Zipper (B-LZ) or Complementary Leucine Zipper (C-LZ) on the cell surface. Alternating doses allow for cellular crosslinking for enhanced accumulation and retention at the site of infarction.

FIGS. 2A-2I are directed to the design of leucine zippers and ZipperCell conjugation. FIG. 2A shows structures of customized synthetic leucine zipper constructs. FIG. 2B shows Coomassie Brilliant Blue stained SDS polyacrylamide gel with ladder, complementary leucine zipper (C-LZ) 10 nM, and base leucine zipper (B-LZ), respectively. FIG. 2C shows three orthogonal pairs of leucine zippers with varying binding affinities of 10, 80, and 200 nM. FIG. 2D is a schematic representation of cellular leucine zipper decoration via heterobifunctional crosslinker (Sulfo SMCC), followed by maleimide-thiol conjugation. FIG. 2E shows Leucine zipper density on cells is controlled by varying the leucine zipper concentration. FIG. 2F shows viability of ZipperCells. FIG. 2G shows detection of leucine zippers on cells as a function of time. Data are presented as mean±SD with *p<0.05, **p<0.01, ****p<0.0001 by one-way ANOVA followed by Tukey's post-test, n=3. FIG. 2H shows the design and sequence of custom synthetic leucine zippers. FIG. 2I is a schematic of major leucine zipper components and customizations.

FIGS. 3A-3G are directed to leucine zipper decoration facilitated crosslinking. FIG. 3A is a schematic of FRET. When bound and excited, AlexaFluor555 labeled B-LZ transfers energy to AlexaFluor647 labeled C-LZ for subsequent emission. FIG. 3B shows FRET spectra of heterodimerizing pairs versus scramble control. FIG. 3C shows FRET efficiency. FIG. 3D shows a monolayer of scramble-decorated cells (DiO lipid dye=green) was seeded and then incubated with a layer of B-LZ (DiI=red) and an additional layer of MSCs decorated with a scrambled peptide (blue=DiD). FIG. 3E shows a monolayer of C-LZ (DiO) followed by 2 additional layers of C-LZ (DiI & DiD). FIG. 3F shows a monolayer of C-LZ (green=DiO) followed by B-LZ decorated cells (red=DiI lipid dye) and then an additional layer of C-LZ cells (blue=DiD). FIG. 3G shows the average thickness of three-dimensional, layer-by-layer cocultures. Data are presented as mean±SD with *p<0.05, **p<0.01, ****p<0.0001 by one-way ANOVA followed by Tukey's post-test, n=3.

FIGS. 4A-4E show that Leucine Zippers maintain normal phenotype in vitro. FIG. 4A shows qRT-PCR of phenotypic MSC marker total RNA expression from unmodified MSCs and ZipperCells seven days after surface decoration. ZipperCells retained their capacity to differentiate into (FIG. 4B) osteoblasts (calcium deposits stained with Alizarin Red S) and (FIG. 4C) adipocytes (lipid droplets stained with Oil Red O). FIG. 4D shows quantification of Oil Red Stain. FIG. 4E shows quantification of Alizarin Red Stain. Differentiation was performed for 21 days and then characterized. Data are presented as mean±SD with *p<0.05, **p<0.01, ****p<0.0001 by one-way ANOVA followed by Tukey's post-test, n=3.

FIGS. 5A-5D show biodistribution of MSCs in mice with MI. FIG. 5A shows representative IVIS images using the IVIS Spectrum in vivo imaging system show DiR-labeled MSC deposition in the infarcted heart. FIG. 5B shows quantification of DiR fluorescence at the infarcted heart. FIG. 5C shows quantification of fluorescence in major organs. FIG. 5D shows Masson's Trichrome staining of whole heart sections. FIG. 5E shows immunofluorescent staining of cryosections of the infarcted heart of mice treated with PBS, unmodified MSCs, and ZipperCells. Cryosections were stained for Troponin I (green), human CD29 (red), and nuclei (DAPI, blue). Scale bar=50 μm. Data are presented as mean±standard deviation. Data are presented as mean±SD with *p<0.05, **p<0.01, ****p<0.0001 by one-way ANOVA followed by Tukey's post-test, n=7.

FIGS. 6A and 6B show long-term leucine zipper expression and cellular retention at the infarct site. FIG. 6A includes representative IVIS images of DiR-labeled MSCs ten days after the initial injection. FIG. 6B shows quantitation of fluorescent cell signal from hearts ten days after the initial injection. Data are presented as mean±SD with *p<0.05, **p<0.01, ****p<0.0001 by one-way ANOVA followed by Tukey's post-test, n=3.

FIGS. 7A-7E illustrates disassembly of accumulated ZipperCells in vitro and in vivo via competition. Coculture of the monolayer of DiO B-LZ & DiI C-LZ 80 nM without (FIG. 7A) and with (FIG. 7B) the addition of 100×C-LZ 10 nM affinity zipper. FIG. 7C shows the quantitation of DiI signal with and without the C-LZ 10 nM affinity competition. FIG. 7D shows representative IVIS images were acquired of the C-LZ 80 nM affinity ZipperCell pair followed by a 1 μg dose of C-LZ 10 nM affinity leucine zipper. FIG. 7E shows quantification of DIR fluorescence at the infarcted heart. Scale Bar=50 μm. Data are presented as mean±SD with *p<0.05, **p<0.01, ****p<0.0001 by one way ANOVA followed by Tukey's post-test, n=3.

FIGS. 8A-8C show that ZipperCells are non-immunogenic. FIG. 8A shows total IgG detection against C-LZ 10 nM in serum. FIG. 8B shows total IgG detection against B-LZ in serum. FIG. 8C shows relative cytokine expression levels of mice the day before surgery (DO) and four hours after the 2nd injection (40 hours post-MI) of PBS, unmodified MSCs, or ZipperCells. Data are presented as mean±SD with *p<0.05, **p<0.01, ****p<0.0001 by one-way ANOVA followed by Tukey's post-test, n=3.

FIG. 9 includes schematic illustrations showing a comparison between the disclosed molecular docking prediction of an established synthetic heterodimerizing leucine zipper pair and x-ray crystallography data. Intermolecular interacting residues are bolded in both the dimer schematics in the top panel and the amino acid nomenclature comparisons in the bottom panel). This shows that the disclosed molecular docking procedure leads to reliable predictions in binding affinity between leucine zippers.

FIGS. 10A and 10B show an illustrative representation of optimal leucine zipper amino acid properties. By mutating amino acids (FIG. 10B) to enhance hydrophilicity of positions c, f or b, the Non-Interacting Surface (NIS) of the leucine zipper chain was altered. By mutating amino acids to increase the charged and hydrophobic content within positions e and g or a and d, respectively, it is possible, as demonstrated herein, to alter the Interfacial contacts (IC) and therefore the strength of interactions between leucine zipper chains (FIGS. 10A and 10B).

FIGS. 11A and 11B show select leucine zipper mutant protein sequences with mutated amino acids shown in highlights (FIG. 11A). By incorporating these mutations, the resulting molecular docking predictions show enhancement in the number of intermolecular interacting residues (FIG. 11B; shown by darkened shades of branching and darkened chain components).

FIGS. 12A and 12B show binding curves (FIG. 12A) used to determine relative binding affinities (FIG. 12B) of the top four mutated protein sequences to a previously established leucine zipper sequence (SynZip2). These show that the disclosed modeling and subsequent mutational incorporation produce functional proteins with enhanced binding affinity.

FIGS. 13A and 13B show the cardiac accumulation of fluorescently labeled exosomes (Exo), also referred to as extracellular vesicles, injected via tail vein into a mouse model of permanent ligation of the left coronary artery (LCA). Female C57BL/6 mice 10 to 12 weeks of age were injected with 3 doses of 1.33e⁹ DiR labeled exosomes, given every 12 hours for a total of 4e⁹ exosomes (also known as EVs) per mouse. Exosomes were isolated from C57BL/6 murine, bone-marrow derived mesenchymal stem cells. (FIG. 13A) Ex vivo IVIS imaging of mouse hearts isolated after 12 hours after the final dose, 48 hours after the first injection (60 hours post-MI). Scramble, Zip1, and NIS1 exosomes were surface decorated with heterodimerizing leucine zippers A scramble control peptide containing an identical amino acid content without a leucine heptad was included as a control. (FIG. 13B) This shows that MSC exosomes surface-decorated with novel leucine zipper sequences, specifically NIS1, facilitate about 7-fold greater in vivo accumulation when compared to unmodified exosome control. Data are mean±sd. **P<0.01 by one way ANOVA. N=4.

FIGS. 14A-14D show echocardiographic parameters of cardiac function of mice with a myocardial infarction after treatment with Zipper-modified MSC exosomes as determined via parasternal short axis view. Myocardial infarction was induced in female C57BL/6 mice 10 to 12 weeks of age by permanent ligation of the LCA. Mice were injected with 3 doses of 1.33e⁹ DiR labeled exosomes, given every 12 hours for a total of 4e⁹ exosomes per mouse. Exosomes were isolated from C57BL/6 murine, bone-marrow derived mesenchymal stem cells. Scramble, Zip1, and NIS1 exosomes were surface decorated with heterodimerizing leucine zippers. A scramble control peptide containing an identical amino acid content without a leucine heptad was included as a control. This shows that the disclosed protein sequences facilitate a highly statistical improvement in cardiac function by measures of Ejection Fraction (FIG. 14A; EF), Fractional Shortening (FIG. 14B; FS), Left Ventricle End Systolic Diameter (FIG. 14C; LVESD), and Left Ventricle End Diastolic Diameter (FIG. 14D; LVEDD). Data are mean±sd. **P<0.01 by one way ANOVA. N=8-10.

FIGS. 15A-15B depict the further characterization of high-affinity leucine zippers as disclosed herein. FIG. 15A shows binding affinity of the top HiA C-LZ (squares) compared with the C-LZ (circles) as assessed by ELISA.

FIG. 15B is a schematic representation of the increased number of intermolecular interactions of the most stable, high binding mutant protein, NIS1 (HiA C-LZ).

FIGS. 16A-16D are directed to the characterization of MSC Zippersomes. FIG. 16A is a schematic representation of cellular leucine zipper decoration using the heterobifunctional crosslinker (Sulfo SMCC), followed by maleimide-thiol conjugation. FIG. 16B shows representative TEM images of unmodified EVs and various Zippersomes. FIG. 16C shows a patterned monolayer of B-LZ incubated with fluorescently labeled unmodified EVs or HiA Zippersomes. FIG. 16D shows calculations of contact surface length and cluster perimeter length based on TEM images (n=20). Data are presented as mean±SD with *p<0.05, **p<0.01, ****p<0.0001 by one-way ANOVA followed by Tukey's post-test.

FIGS. 17A and 17B are directed to cellular uptake of Zippersomes by cardiac cells. FIG. 17A shows cellular uptake and binding of DiI-labeled EVs by four representative cardiac cell types: (1) H9C2 (rat cardiomyocytes), (2) HUVECs (human umbilical vein endothelial cells), (3) HCFs (human cardiac fibroblasts), and (4) RAW 264.7 (murine macrophages). DiO was used to label cell membranes prior to incubation. Blue=DAPI, white=DiO, red=DiI. FIG. 17B shows quantification of total fluorescence in each cell line (n=3). Data are presented as mean±SD with *p<0.05, **p<0.01, ****p<0.0001 by one-way ANOVA followed by Tukey's post-test.

FIGS. 18A-18D show Zippersome accumulation and retention in vivo. FIG. 18A is schematic and experimental timeline of alternating Zippersome therapy. FIG. 18B shows representative IVIS images using the IVIS Spectrum in vivo imaging system show DiR-labeled EV deposition in the infarcted heart. FIG. 18C shows quantification of DiR fluorescence in the infarcted heart. FIG. 18D shows quantification of DiR fluorescence in major organs. Scale bar=50 μm. Data are presented as mean±SD with *p<0.05, **p<0.01, ****p<0.0001 by one-way ANOVA followed by Tukey's post-test; n=8-10.

FIGS. 19A-19D show long term EV retention in the infarcted myocardium. FIG. 19A shows representative IVIS images of DiR-labeled EVs 21 days after the initial injection. FIG. 19B shows the quantitation of fluorescent EV signal from hearts 21 days after the initial injection. FIG. 19C shows H&E staining and FIG. 19D shows CD45 staining of liver tissues 21 days after injections. Data are presented as mean±SD with *p<0.05, **p<0.01, ****p<0.0001 by one-way ANOVA followed by Tukey's post-test, n=4.

FIGS. 20A-20D shows that Zippersome treatment improves cardiac function and reduces infarct size. FIG. 20A includes representative 2D M-mode echocardiography images at study completion (Day 21). Parasternal short-axis view. FIG. 20B shows Masson's Trichrome staining was used to evaluate LV fibrotic area. FIG. 20C shows longitudinal assessment of LV ejection fraction (EF, %), fractional shortening (FS, %), and left ventricular end diameter in systole (LVESD, mm) and diastole (LVEDD, mm). FIG. 20D shows percentage of fibrotic LV in PBS, unmodified EV, and Zippersome hearts. Data are presented as mean±standard deviation. Data are presented as mean±SD with *p<0.05, **p<0.01, ****p<0.0001 by one-way ANOVA followed by Tukey's post-test, n=8-10.

FIGS. 21A-21E show spatial analysis of treated infarcted hearts. Representative masson trichrome and multiplex fluorescence staining of (FIG. 21A) PBS and (FIG. 21B) HiA Zippersome heart sections. Violin plots of numbers of each cell type within the remote (FIG. 21C), border (FIG. 21D), and infarct zones (FIG. 21E) in PBS and HiA Zippersome samples. Data are presented as mean±SD with *p<0.05, **p<0.01, ****p<0.0001 by one-way ANOVA followed by Tukey's post-test, n=3.

### DETAILED DESCRIPTION

The presently disclosed subject matter now will be described more fully hereinafter, in which some, but not all embodiments of the presently disclosed subject matter are described. Indeed, the presently disclosed subject matter can be embodied in many different forms and should not be construed as limited to the embodiments set forth herein; rather, these embodiments are provided so that this disclosure will satisfy applicable legal requirements.

### I. Definitions

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of a composition, dose, sequence identity (e.g., when comparing two or more nucleotide or amino acid sequences), mass, weight, temperature, time, volume, concentration, percentage, etc., is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

The term “comprising”, which is synonymous with “including” “containing” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

The term “gene” refers broadly to any segment of DNA associated with a biological function. A gene can comprise sequences including but not limited to a coding sequence, a promoter region, a cis-regulatory sequence, a non-expressed DNA segment that is a specific recognition sequence for regulatory proteins, a non-expressed DNA segment that contributes to gene expression, a DNA segment designed to have desired parameters, or combinations thereof. A gene can be obtained by a variety of methods, including cloning from a biological sample, synthesis based on known or predicted sequence information, and recombinant derivation of an existing sequence.

The term “substantially identical”, as used herein to describe a degree of similarity between nucleotide sequences, peptide sequences and/or amino acid sequences refers to two or more sequences that have in one embodiment at least about least 60%, in another embodiment at least about 70%, in another embodiment at least about 80%, in another embodiment at least about 85%, in another embodiment at least about 90%, in another embodiment at least about 91%, in another embodiment at least about 92%, in another embodiment at least about 93%, in another embodiment at least about 94%, in another embodiment at least about 95%, in another embodiment at least about 96%, in another embodiment at least about 97%, in another embodiment at least about 98%, in another embodiment at least about 99%, in another embodiment about 90% to about 99%, and in another embodiment about 95% to about 99% nucleotide identity, when compared and aligned for maximum correspondence, as measured using a sequence comparison algorithm or by visual inspection.

As used herein, the term “subject” refers to an individual (e.g., human, animal, or other organism) to be assessed, evaluated, and/or treated by the methods or compositions of the presently disclosed subject matter. Subjects include, but are not limited to, mammals (e.g., murines, simians, equines, bovines, porcines, canines, felines, and the like), and includes humans. As used herein, the terms “subject” and “patient” are used interchangeably, unless otherwise noted.

As used herein, the terms “effective amount” and “therapeutically effective amount” are used interchangeably and refer to the amount that provides a therapeutic effect, e.g., an amount of a composition that is effective to treat or prevent pathological conditions in a subject.

As used herein, the term “adjuvant” as used herein refers to an agent which enhances the pharmaceutical effect of another agent.

A “compound”, as used herein, refers to any type of substance or agent that is commonly considered a chemical, drug, or a candidate for use as a drug, as well as combinations and mixtures of the above. The term compound further encompasses molecules such as peptides and nucleic acids.

As used herein, a “derivative” of a compound refers to a chemical compound that can be produced from another compound of similar structure in one or more steps, such as in replacement of H by an alkyl, acyl, or amino group.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “modulate”, as used herein, refers to changing the level of an activity, function, or process. The term “modulate” encompasses both inhibiting and stimulating an activity, function, or process.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in an animal. In some embodiments, a pharmaceutically acceptable carrier is pharmaceutically acceptable for use in a human.

The term “standard”, as used herein, refers to something used for comparison. For example, it can be a known standard agent or compound which is administered or added to a control sample and used for comparing results when measuring said compound in a test sample. Standard can also refer to an “internal standard”, such as an agent or compound which is added at known amounts to a sample and is useful in determining such things as purification or recovery rates when a sample is processed or subjected to purification or extraction procedures before a marker of interest is measured.

The term “symptom”, as used herein, refers to any morbid phenomenon or departure from the normal in structure, function, or sensation, experienced by the patient and indicative of disease. In contrast, a sign is objective evidence of disease. For example, a bloody nose is a sign. It is evident to the patient, doctor, nurse and other observers.

As used herein, the term “treating” includes prophylaxis of the specific disorder or condition, or alleviation of the symptoms associated with a specific disorder or condition and/or preventing or eliminating said symptoms. A “prophylactic” treatment is a treatment administered to a subject who does not exhibit signs of a disease or exhibits only early signs of the disease for the purpose of decreasing the risk of developing pathology associated with the disease.

A “therapeutic” treatment is a treatment administered to a subject who exhibits signs of pathology for the purpose of diminishing or eliminating those signs.

### II. Detailed Discussion of Heterodimerizing Leucine Zippers for Forming Cross-Link Scaffold-Like Materials as a Drug Delivery Systems and Methods

The accumulation and retention of cells, cell-based therapeutics, drug carriers, delivery vehicles, nanocarriers, therapeutics, biologics, nucleic acids, etc. can be enhanced at the infarct site by surface-decorating them with heterodimerizing leucine zippers that accumulate at the site of MI and cross-link into scaffold-like materials. Unlike conventional delivery strategies, where cells or carriers can no longer accumulate once the targeted areas at infarct sites are saturated and occupied, each dose of cells in the disclosed drug delivery platform will serve as a capturing surface for the next dose of cells. This will dramatically amplify the accumulation of cells at the desired sites in a layer-by-layer fashion and allow adjustment of the concentration and type of therapy in subsequent doses.

Leucine zippers are a class of protein dimerization domains with a wide range of binding affinities from low picomolar to micromolar ranges and are therefore ideal for use in the disclosed platform. These α-helical proteins are characterized, in some embodiments, by a series of leucines spaced 7 residues apart. Through a series of hydrophobic, hydrophilic, and ionic interactions, dimerization between zippers is mediated. While present endogenously, specifically designed synthetic leucine zippers are stable at a wide range of pH values and salt concentrations and can survive the acidic environments of infarcts, and many other physiological conditions. Depending on their structure, specific zippers do not homodimerize but instead form heterodimers with specific counterparts which minimizes off-target crosslinking.

Disclosed herein is a novel platform to improve cell accumulation and retention at the site of myocardial infarction. Heterodimerizing zippers were selected to achieve selective crosslinking while preventing self-aggregation via homodimerization. The ability of leucine zipper decorated cells, termed ZipperCells, to form stable, but reversible physical crosslinking under in vitro conditions were then characterized and tested. Next, it was assessed whether this crosslinking allows for enhancement in accumulation and prolonged retention under systemic administration in a preclinical model of MI. Finally, the capacity of ZipperCells to be exchanged or outcompeted, which would make them ideal for use in minimally invasive cell therapy, was tested.

The term “cross-link” is used herein to refer to any suitable method, approach, or system for attaching the disclosed leucine zippers to a surface of a cell. Thus, in some embodiments, “cross-linked” and “attached” are used interchangeably. For example, and without limitation, such “cross-linking” or “attaching” can refer to:
(1) Any crosslinkers or chemical approaches capable of attaching leucine zippers to the surface of cells, extracellular vesicles, nanocarriers, therapeutics, etc. Examples of such crosslinkers include, but are not limited to: AMAS (N-α-maleimidoacet-oxysuccinimide ester), BMPS (N-β-maleimidopropyl-oxysuccinimide ester), EMCS (N-ε-malemidocaproyl-oxysuccinimide ester), GMBS (N-γ-maleimidobutyryl-oxysuccinimide ester), LC-SMCC (succinimidyl 4-(N-maleimidomethyl)cyclohexane-1-carboxy-(6-amidocaproate)), LC-SPDP (succinimidyl 6-(3 (2-pyridyldithio) propionamido) hexanoate), MBS (m-maleimidobenzoyl-N-hydroxysuccinimide ester), PEG12-SPDP (PEGylated, long-chain SPDP crosslinker), PEG4-SPDP (PEGylated, long-chain SPDP crosslinker), SM(PEG)2 (PEGylated SMCC crosslinker), SM(PEG)24 (PEGylated, long-chain SMCC crosslinker), Sulfo-SMPB (sulfosuccinimidyl 4-(N-maleimidophenyl) butyrate), Sulfo-MBS (m-maleimidobenzoyl-N-hydroxysulfosuccinimide ester);
(2) Any genetic approaches to display leucine zippers on the surface of cells, extracellular vesicles, nanocarriers, therapeutics, etc. For example, leucine zippers can be genetically fused to membrane proteins found on cells, extracellular vesicles, nanocarriers, therapeutics. Examples of such proteins are tetraspanins, integrins, cadherins, selectins, membrane receptors, and others. Leucine zippers can be chemically modified to contain biotin for binding to streptavidin that are displayed on the surface of cells, extracellular vesicles, nanocarriers, and therapeutics;
(3) Physical approaches that mediate absorption or attachment of leucine zippers to the surface of cells, extracellular vesicles, nanocarriers, therapeutics, etc. Leucine zippers can be chemically conjugated to lipids of different tail lengths. These lipids can provide insertion into the surface/lipid bilayer of cells, extracellular vesicles, and nanocarriers. Leucine zippers can also be fused to hydrophobic peptides or amphiphilic peptides for insertion into the surface/lipid bilayer of cells, extracellular vesicles, and nanocarriers.

Thus, in some embodiments, provided herein is a non-invasive, in situ forming depot for delivery of a therapeutic agent, comprising one or more heterodimerizing, synthetic leucine zippers for physical crosslinking mediated by competition-based dimerization, and a therapeutic agent, wherein the one or more heterodimerizing, synthetic leucine zippers form a self-assembling depot of the therapeutic agent at a target site in vivo. In some aspects, the heterodimerizing, synthetic leucine zippers amplify an available binding area at a target site in vivo in a layer-by-layer fashion. In some embodiments, the therapeutic agent comprises an affinity or attraction to a target site in vivo, optionally comprising a targeting ligand on a surface of the therapeutic agent, to enhance accumulation of the therapeutic agent at the target site. In some embodiments, the non-invasive, in situ forming depots for delivery of a therapeutic agent achieve significantly enhanced retention and accumulation of the therapeutic agent at the target site. In some embodiments, the therapeutic agent comprises a cell, optionally a stem cell, a nanoparticle, a microparticle, a drug carrier, a small molecule, a pharmaceutical, a biotherapeutic, a biologic, and combinations thereof. In some embodiments, the therapeutic agent comprises a stem cell, wherein the stem cell comprises a targeting ligand on a surface of the stem cell. In some embodiments, the therapeutic agent comprises a mesenchymal stem cell with a natural ability to migrate to sites of inflammation in vivo. In some embodiments, the target site is an infarct site, wherein the therapeutic agent comprises a stem cell. In some embodiments, the stem cell maintains a regenerative wound healing property and/or capacity to differentiate after deposition at the target site.

In some embodiments, the in situ forming depots are configured to be sequentially administered to a subject, whereby one or more layers of the in situ forming depots can be formed at a target site. In some embodiments, the one or more heterodimerizing, synthetic leucine zippers facilitate physical multivalent crosslinking and therefore retention and accumulation of the therapeutic agent at the target site. In some embodiments, each layer serves as an additional capturing surface for a subsequent dose of a non-invasive, in situ forming depot. In some embodiments, the non-invasive, in situ forming depots are configured to be sequentially administered to a subject, whereby each sequential administration is configured to provide a different therapeutic agent and/or a different dosage of the therapeutic agent. In some embodiments, the non-invasive, in situ forming depots are configured for a layer-by-layer dosing strategy. In some embodiments, the non-invasive, in situ forming depots are configured for intravenous administration to a subject, optionally for any other suitable form/route of administration, including but not limited to subcutaneous, intramuscular, local, intracranial, intraarterial. In some embodiments, multivalent crosslinking of the one or more leucine zippers increases resistance to venous washout, allowing the therapeutic agent to persist at the target site long term.

In some embodiments, the non-invasive, in situ forming depots, including the therapeutic agent, are configured to persist at the target site for at least about 5 days, about 10 days, about 15 days, about 20 days, about 30 days, about 50 days, about 100 days, or more. In some embodiments, the non-invasive, in situ forming depots, including the therapeutic agent, are configured to deposit at the target site at a concentration of at least about 2 times, about 5 times, about 10 times, about 20 times, about 50 times, or more, as compared to a therapeutic agent without the non-invasive, in situ forming depots. In some embodiments, the one or more leucine zippers comprise an amino acid or nucleotide sequence of any of SEQ ID NOs. 1-8, or a variant thereof substantially identical to any of SEQ ID NOs. 1-8, optionally wherein the variant has a homology of at least about 75%, 80%, 85%, 90%, 91%, 92%, 93%, 94%, 95%, 96%, 97%, 98%, 99% or more as compared to any of SEQ ID NOs. 1-8. In some embodiments, the non-invasive, in situ forming depots are configured to be disassembled via competition-mediated disassembly.

Also provided herein in some embodiments are methods of treating, ameliorating and/or preventing a condition in a subject, the method comprising providing a subject in need of treatment, amelioration and/or prevention of a condition, and administering to the subject a non-invasive, in situ forming depot of any of the above claims. In some embodiments, the subject is a human subject, optionally wherein the subject is suffering from myocardial infarction or is susceptible to suffering from the same, optionally wherein the subject is suffering from an inflammatory condition, ischemia, cancer, arthritis, joint disease, limb ischemia, etc. In some embodiments, the subject is administered sequential doses of the non-invasive, in situ forming depot, optionally wherein the therapeutic agent and/or dosage varies between the sequential doses.

Provided herein are leucine zippers comprising an amino acid sequence of any of SEQ ID NOs. 1, 3, 5 or 7, or a substantially identical variant thereof, optionally wherein the variant has a homology of at least about 75%, 80%, 85%, 90%, 91%, 92%, 93%, 94%, 95%, 96%, 97%, 98%, 99% or more as compared to any of SEQ ID NOs. 1, 3, 5 or 7. In some embodiments, provided herein are leucine zippers comprising an amino acid sequence of any of SEQ ID NOs. 1, 3, 5 or 7, or a substantially identical variant thereof, wherein amino acids 22-47 of SEQ ID NOs. 1, 3, 5 or 7 are substantially conserved. In some embodiments, provided herein are leucine zippers comprising an amino acid sequence of any of SEQ ID NOs. 1, 3, 5 or 7, or a substantially identical variant thereof, wherein amino acids 22-47 of SEQ ID NOs. 1, 3, 5 or 7 are substantially conserved, and wherein the remainder of SEQ ID NOs. 1, 3, 5 or 7 has a homology of at least about 75%, 80%, 85%, 90%, 91%, 92%, 93%, 94%, 95%, 96%, 97%, 98%, 99% or more as compared to any of SEQ ID NOs. 1, 3, 5 or 7. In some embodiments amino acids 22-47 of SEQ ID NOs. 1, 3, 5 or 7 is considered the conserved region or the functional region, wherein conserved regions or conserved peptide typically undergo fewer amino acid replacements, or are more likely to substitute amino acids with similar biochemical properties. Within a sequence, amino acids important for folding, structural stability, or that form a binding site may be more highly conserved.

Provided are also leucine zippers comprising nucleotide sequence of any of SEQ ID NOs. 2, 4, 6 or 8, or a substantially identical variant thereof, optionally wherein the variant has a homology of at least about 75%, 80%, 85%, 90%, 91%, 92%, 93%, 94%, 95%, 96%, 97%, 98%, 99% or more as compared to any of SEQ ID NOs. 2, 4, 6 or 8. In some aspects, the leucine zipper sequences display mutated amino acids that are primarily positioned within the center of the leucine zipper chain to obtain enhanced heterodimer stabilization.

In some aspects provided is a pharmaceutical composition comprising a leucine zipper-based therapeutic in combination with a stem cell therapeutic, wherein one or more stem cells in the stem cell therapeutic are tagged with a leucine zipper. In some embodiments, the leucine zipper-based therapeutic comprises a leucine zipper selected from a library of heterodimerizing, synthetic leucine zippers for physical crosslinking mediated by competition-based dimerization. The pharmaceutical composition can be configured for non-invasive, targeted, systemic administration in a subject. The pharmaceutical composition can provide stem cell as mediators of wound healing, optionally wherein the stem cells are configured to self-renew, immunomodulate, and/or differentiate into a variety of cell types for regenerative medicine.

### III. Detailed Discussion of Heterodimerizing Leucine Zippers with Extracellular Vesicles (EVs) for Forming Cross-Link Scaffold-Like Materials as a Therapeutic Formulations

Despite the widespread use of stem cells for the treatment of myocardial infarction (MI), acellular approaches may pose fewer barriers to clinical translation and therefore should be considered as therapeutic options. Extracellular vesicles (EVs), in some embodiments defined as lipid vesicles secreted by cells, can provide a middle ground between cellular and synthetic approaches. EVs contain many of the biomaterials typically found in their parent cells including proteins, RNAs, growth factors, and chemokines, but, in contrast to cells, are easier to handle, store, and ship. Their small size, physicochemical properties, and low immunogenicity permit deep tissue penetration, facilitate intercellular communication, and mediate tissue regeneration. While studies have directly injected EVs into the myocardium to deliver them to the target site, this approach is highly invasive, can cause arrhythmias, and can have deleterious effects on cardiac function. Other therapies attempt to circumvent this shortcoming by administering EVs systemically; however, rapid washout and poor accumulation limit their efficacy. Therefore, there remains a significant need for approaches that can increase EV exposure within the infarct site, preferably non-invasively.

Inefficient delivery to the infarct site is an issue not limited to EVs but other nanocarriers as well. Though widely used, nanoparticles often suffer from poor homing and rapid, non-specific biodistribution. For example, micelles and liposomes can accumulate in the infarcted myocardium for approximately one day but cannot be detected long-term. Studies demonstrated that less than 0.5% of systemically administered nanoparticles (20 nm-2 μm) are retained after 30 minutes. Other studies have shown that nanoparticles that serve as drug carriers can prolong drug retention, but not long-term, i.e. for at least several days. For example, PLGA nanoparticles loaded with IGF-1 could prolonged delivery for at least 24 hours but not more than three days. Many strategies to enhance the accumulation of nanocarriers or EVs at the infarct site focus on improving nanoparticle targeting or use of additional nanomaterials, such as hydrogels and nanocomplexes. However, these approaches can be limited by low loading efficiency and the need for additional materials. Therefore, an effective and efficient strategy at the nanoscale level would be highly desirable to significantly advance the field.

As disclosed and shown herein, mesenchymal cells (MSCs) decorated with either base leucine zippers (B-LZ) or their heterodimerizing proteins, complementary leucine zippers (C-LZ), can crosslink in situ to form a cellular depot at the infarct site. Characteristically, leucine zippers are composed of heptad sequences, with leucines every seven residues. Their stable but reversible binding is facilitated by a combination of hydrophobic, hydrophilic, and electrostatic interactions. To improve the utility of this protein-based system, it was further investigated, and is disclosed herein, whether strengthening leucine zipper binding by improving the positions of each intermolecular interaction within the hetero-dimer in the next generation of high-affinity leucine zippers can (i) enhance vesicle cross-linking; and (ii) in vivo, under systemic flow conditions, better resist venous washout and promote cellular internalization. By improving the positions of each of these interactions, provided herein are increased intermolecular interactions within the dimer and improved binding strength. To enable precise crosslinking and enhance the targetable surface areas, the strategic utilization of heterodimeric leucine zippers was exclusively employed, rather than homo-dimers. This deliberate choice ensures controlled capturing of each subsequent dose of EVs by every administered dose. This intentional approach effectively safeguards against unintended aggregation and maintains optimal functionality.

Disclosed herein is the development of the next-generation delivery platform of therapeutics to infarcted myocardium, and other conditions. Disclosed is the first evidence for a non-invasive, in situ crosslinking of small EVs to mediate enhanced and prolonged retention in the infarcted myocardium. This disclosure further assesses the effect of a novel, high-binding affinity leucine zipper on the in vivo crosslinked drug depot. To achieve this, the EVs were first decorated with leucine zippers, termed Zippersomes, which were then assessed for their ability to crosslink, heterospecifically, in vitro. Next, it was examined whether this crosslinking enhanced EV accumulation and retention in a murine model of MI. Finally, the effect of Zippersome treatment on cardiac function and infarct size was tested.

### EXAMPLES

The following examples are included to further illustrate various embodiments of the presently disclosed subject matter. However, those of ordinary skill in the art should, in light of the present disclosure, appreciate that many changes can be made in the specific embodiments which are disclosed and still obtain a like or similar result without departing from the spirit and scope of the presently disclosed subject matter.

### Materials and Methods for Examples 1-10

Cell Culture: Human Bone-marrow derived MSCs were obtained from the American Type Culture Collection (ATCC PCS-500-012) and cultured with DMEM/F12 medium supplemented with 10% FBS (Gibco™, Gaithersburg, MD). Cells were maintained in hypoxic conditions (5% CO2, 5% O2) for 24 hours prior to studies.

Protein Production and Purification: The customized leucine zipper sequences were cloned into a pET21a vector (Novagen, Burlington, MA) and expressed in BL-21 (DE3) cells (Lucigen, Middleton, WI). Bacterial cultures were grown overnight at 37° C. in 2×YT (Fisher Scientific, Waltham, MA) medium and ampicillin. For large-scale production, a 10 L 2×YT culture was inoculated with 1% of the overnight culture and grown to a high density at 37° C. for 8 hours. Protein expression was induced with 20 g/L alpha lactose. After overnight incubation at 24° C., the cells were harvested 16 h post-induction by centrifugation. The cell pellet was resuspended in lysis buffer containing 50 mM Tris, 100 mM KCl, 1 mM DTT, 10% glycerol, and 100 μg/ml lysozyme, pH 8.0. The cell suspension was sonicated and then centrifuged at 17,000×g for 40 minutes at 4° C., after which Ni-NTA Resin (Gbiosciences, St. Louis, MO) was added to the solution. The beads were incubated with the solution and the protein was isolated by immobilized metal affinity chromatography (IMAC). Endotoxin removal was performed using Pierce High-Capacity Endotoxin Removal Resin (Thermofisher) and validated using the Pierce LAL Chromogenic Endotoxin Kit Quantitation Kit (Thermofisher).

Enzyme-linked immunosorbent assay (ELISA): Nunc MaxiSorp 96-well plates were coated with Leucine Zippers at 2.5 μg/mL and incubated overnight at 4° C. The plate was thoroughly washed with 0.1% TBS-Tween (TBST). The wells were washed with 0.1% PBST and then blocked with BSA blocking buffer for 1 h at room temperature. Then incubated with 3.3 μM complimentary leucine zipper at 37° C. for 2 hours before washing. Washes were followed by the addition of 100 μL of Anti-FLAG horseradish peroxidase (HRP)-linked monoclonal antibody (mAb, #A8592, Sigma Aldrich, St. Louis, MO) for 1 h at room temperature with rocking. The wells were washed with 0.1% PBST followed by the addition of TMB-ELISA substrate. After 10 minute incubation, 2 M H2SO4 was added to stop the reaction. The absorbance was measured at 450 nm and 570 nm with a SpectraMax M5 plate reader (Molecular Devices, Sunnyvale, CA).

Protein-Cell Conjugation: Cells were trypsinized, washed, and resuspended in HBSS. At a concentration of 1.0E⁶ cells/ml cells were incubated with 2.5 μM DiR dye and incubated with sulfosuccinimidyl 4-(N-maleimidomethyl)cyclohexane-1-carboxylate (Sulfo-SMCC). at a final concentration of 24 μg/ml for 30 minutes at 37° C. To reduce protein for conjugation, proteins were incubated with 1 mM tris(2-carboxyethyl) phosphine (TCEP) at room temperature for 20 minutes. After washing cells via centrifugation, the protein was added to cells and incubated at room temperature for 30 minutes before final washing and resuspension.

Fluorescent Protein Labeling: For general protein labeling, protein solutions were labeled with fluorescein isothiocyanate. For Förster resonance energy transfer (FRET), proteins were labeled using N-hydroxysuccinimide ester amine chemistry with Alexa Fluor™ 555 C2 Maleimide (Invitrogen) for the base leucine zipper or AF657 NHS Ester (Invitrogen) for complementary leucine zippers. Protein samples were first reduced using 5 mM TCEP. Then proteins were mixed with 10-fold molar excess dye and incubated at room temperature for 1 hour. After labeling, the free dye was removed using a PD-10 column. Labeling efficiency (1:1 ratio) was ensured by measuring the absorbance at 555 nm (AF555, ε=158,000 M⁻¹ c⁻¹) or 650 nm (AF647, ε=270,000 M⁻¹ c⁻¹) in PBS for the dye and 280 nm in PBS for the protein.

Computational Modeling: Computational modeling was used to predict the number of leucine zippers required per cell, to allow for stable network formation by overcoming the shear forces due to blood flow. The model simulated the hierarchical microvasculature in mice^{[20]} with an inlet flow rate of 0.3 ml/minute^{[21]} at the arterial side and −120 mmHg pressure at the venous side.^{[22]} At the central infarct region, the permeability of the leaky microvessels was governed by the Kozeny-Carman permeability model^{[23,24]} (cell radius (r_{c})=10 μm and porosity (ε)=40%^{[23]}). Within each layer, the shear forces (

$${F}_{s} = τ \times π {r}_{c}^{2} \qquad ( 1 )$$

where τ is shear stress) were assumed to act obliquely on each cell. The force required to dissociate the bound proteins

$${F}_{{b}^{=}} R ( {K}_{D} ) / {l}_{t} \qquad ( 2 )$$

was determined as a function of binding affinity (K_{D}) and the length of the crosslinked chain between the cells

$${( {l}_{t} = {l}_{z} + 2 {l}_{s p} + 2 {l}_{c} )}^{[ 26 , 27 ]} \qquad ( 3 )$$

where I_{z} is zipper length (7.5 nm), I_{c} is the crosslinker length (0.7 nm) and Iₛₚ is the average cell surface protein length (2.2 nm). The number of proteins required on the surface of each cell was

$${N}_{p} = {F}_{s} / ( {F}_{b} \times {f}_{s a} ) \qquad ( 4 )$$

$$where$$

$${f}_{s a} = {l}_{z}^{2} / {r}_{c}^{2} \qquad ( 5 )$$

is the fraction of the cell surface area participating in dimerization.^{[25]} The number of bonds (N_{b}) needed to overcome the viscous shear forces was, therefore,

$${N}_{b} = {F}_{s} / {F}_{b} . \qquad ( 6 )$$

For zipper dimerization at 200 nM, 80 nM, and 10 nM binding affinity (K_{D}), the number of zippers (Nₚ) required was calculated as

$$( {N}_{p} = {N}_{b} / {f}_{sa} ) \qquad ( 7 )$$

where the required on the surface of each cell (fₛₐ) was approximated by

$${( {f}_{sa} = {l}_{p}^{2} / {r}_{c}^{2} )}^{[ 25 ]} . \qquad ( 8 )$$

Protein Dilution Study: ZipperCells were generated as described above. Cells were seeded at a density of 10,000 cells per well of a 96 well plate in triplicate. The first time point was recorded 12 hours after plating. Cells were washed and fixed with 4% paraformaldehyde (PFA) for 10 minutes. Cells were then incubated with 100 μl of 1 ug/ml Hoechst 3342 to normalize via cell number. Next, cells were treated with blocking buffer (1% BSA in PBS) for 1 h at 37° C. Wells were incubated with diluted Anti-Flag HRP antibody for 30 minutes at 37° C. and washed with PBS. Finally, cells were incubated with 3,3′,5,5′-Tetramethylbenzidine (TMB) for 15 minutes before measuring absorbance at 650 nm on the plate reader. Hoechst fluorescence (361/486 nm) signal was also measured for quantification of cell number. This was repeated daily for 10 days.

Cell Viability Assay (MTS): ZipperCells were prepared as described above and plated at a density of 10,000 cells per well of a 96 well plate in triplicate along with unmodified MSCs. 3-(4,5-Dimethylthiazol-2-yl)-5-(3-carboxymethoxyphenyl)-2-(4-sulfophenyl)-2H-tetrazolium (MTS) reagent (MTS Reagent Powder, Promega Corporation Cat. #G1111) was prepared according to the manufacturer's instruction. Wells were incubated for 12 hours before recording the first time point. MTS solution was added to each well at a final concentration of 0.33 mg/ml. Cells were incubated for 2 hours at 37° C. before measuring absorbance at 490 nm.

FRET Assay to Measure Heterodimerization: The fluorescence emission of N-terminally AF647 labeled leucine zippers was measured alone and, when mixed with C-terminally Alexa Fluor 555-labeled partners, in triplicate. Samples (100 μL) were mixed at 100 nM concentration of each protein in PBS pH 7.4, 5 mM TCEP, allowed to incubate for 2 h at 37° C., and then equilibrated for 1 h at room temperature. Samples were excited at 555 nm, and emission spectra were monitored from 400 nm to 750 nm at 25° C. Samples were assayed in 96-well black plates (Corning) using a Synergy H1 plate reader (Winooski, VT). FRET efficiency was calculated using the following formula^{[28]}:

$$FRET efficiency = 1 - \frac{{emission ( 580 nm )}_{mix}}{{emission ( 580 nm )}_{donor}} \qquad ( 9 )$$

### Reverse Transcription-Polymerase Chain Reaction (RT-PCR) of MSC Phenotypic Markers:

ZipperCells and unmodified MSCs were plated at a density of 35,000 cells/cm². Seven days later, the gene expression profiles of unmodified MSCs and ZipperCells were quantified by real-time polymerase chain reaction (qPCR). Briefly, harvested cells were fixed in TRIzol, and RNA was extracted from the homogenized cell lysate through a series of rinse, elution, and centrifugation steps. The RNA samples were then reverse transcribed into cDNA using ProtoScript II First Strand cDNA Synthesis Kit reagents (New England Biolabs, Ipswich, MA) following the manufacturer's instructions. In the differentiation studies, the gene expression of interest was determined using iTaq Universal SYBR Green Supermix (Hercules, MA). Five positive MSC phenotypic markers and five negative MSC phenotypic markers were examined^{[29,30]}. The fluorescent signals were amplified and detected using a QuantStudio 3 sequence detector (Applied Biosystems). The cycle threshold (Ct) value for each sample was averaged from triplicates. A 2^{-ΔCt} approach was used where the fluorescent signals were normalized to the corresponding housekeeping gene (glyceraldehyde-3-phosphate dehydrogenase (GAPDH)).

In vitro MSC Differentiation: Cells were plated at a density of 5×10³ cells/cm² in a 12-well plate. When the cells were 60% confluent, osteogenic differentiation was initiated by replacing the complete growth medium with the osteogenic or adipogenic medium.^{[31]} The osteogenic medium was composed of DMEM High Glucose supplemented with 10% FBS, 1% P/S, 100 nM of dexamethasone, 50 M of ascorbic acid, and 10 mM of sodium β-glycerophosphate. The adipogenic medium was composed of DMEM High Glucose supplemented with 10% FBS, 1% P/S, 1 UM dexamethasone, 1 μM insulin, and 200 μM indomethacin. In parallel, the control undifferentiated MSCs were grown in standard complete medium. Cells were grown for 21 days at 37° C. in a humidified 5% CO₂ atmosphere. The medium was changed every 3 days.

Osteogenic Staining: After 21 days, cells were washed with PBS before fixing them with 4% PFA for 15 minutes. Fixative was removed and cells were washed 3× with Deionized (DI water). 1 ml of 1% aqueous Alizarin Red solution (GFS Chemicals) was added to each well and incubated with gentle rocking for 45 minutes. Dye was removed and cells were washed 3× with DI water before imaging using brightfield microscopy.

Adipogenic Staining: After 21 days, cells were washed with PBS before fixing them with 4% PFA for 15 minutes. Fixative was removed and cells were washed 3× with PBS. 1 ml of 0.5% Oil Red solution (Sigma-Aldrich) was added to each well and incubated for 5 minutes at room temperature. Dye was removed and cells were washed 3× with DI water before imaging using brightfield microscopy.

Animals: Female C57BL/6 mice were used (10-12 weeks) for the in vivo studies. All animal procedures were approved by the University of North Carolina at Chapel Hill Institutional Animal Care and Use Committee (IACUC). All methods and experiments were performed in accordance with the U.S. National Institutes of Health Guide for Care and Use of Laboratory Animals. Humane care and treatment of animals were ensured.

Mouse Model of Ischemia/Reperfusion (I/R): All surgeries were performed in the McAllister Heart Institute (MHI) Cardiovascular Physiology and Phenotyping Core. Briefly, mice were anesthetized with isoflurane. A small incision was made under the mandible to visualize the trachea before intubation with a 20-gauge blunt needle and ventilation. A left lateral thoracotomy will expose the heart, and the left coronary artery (LCA) was identified and temporarily occluded with a 7-0 nylon suture for 40 minutes. Reperfusion was confirmed by electrocardiogram (ECG). The thorax was closed in layers (ribs, muscles, and skin)^{[32]}. Mice were provided with analgesics and monitored per protocol.

Biodistribution of MSCs: For biodistribution studies, 500,000 1,1′-Dioctadecyl-3,3,3′,3′-Tetramethylindotricarbocyanine lodide (DiR)-labeled ZipperCells or unmodified MSC, or PBS was administered via tail vein injection into C57BL/6 mice 24 hours after inducing MI. A total of three injections were administered every twelve hours. Twelve hours after the final injection (60 hours post-MI), mice were sacrificed and organs including brain, lung, heart, liver, spleen, and kidneys were collected and weighed. Fluorescent biodistribution was analyzed using the IVIS Spectrum in vivo imaging system (PerkinElmer, Waltham, MA). Average region of interest (ROI) signals were calculated using Living Image 4.5.2 software (PerkinElmer). The data is presented as radiant efficiency/g organ.

Histological Assessment of MSCS in the Infarcted Heart: For histological studies (n=3), 500,000 1,1′-Dioctadecyl-3,3,3′,3′-Tetramethylindotricarbocyanine Iodide (DiR)-labeled ZipperCells or unmodified MSC, or PBS was administered via tail vein injection into C57BL/6 mice 24 hours after inducing MI. A total of three injections were administered every twelve hours. Twelve hours after the final injection (60 hours post-MI), mice were sacrificed, and hearts were collected. The left ventricle was briefly perfused with potassium chloride (30 mM) to arrest the heart in diastole and harvested mouse hearts were embedded in OCT. The infarct was cryosectioned in 10 μm sections.
(1) Immunohistochemistry of MSCS in the Infarcted Heart: Heart cryosections were incubated with anti-Troponin I (Cat #bs-0799R, Bioss USA) and APC anti hCD29 (Cat #102901C0, AAT Bioquest) overnight at 4° C., then stained with a secondary antibody for Troponin I (Cat #A10042, Invitrogen). Sections were then washed 3× with PBST, followed by DAPI mounting using Fluoromount-G, with DAPI (Cat #00-4959-52, Invitrogen). Images were taken with an Olympus FV3000RS and analyzed using ImageJ.
(2) Masson's Trichrome Staining: Masson's staining was performed using a kit (Sigma-Aldrich, Burlington, MA). After cryosectioning and immediate fixation in 95% ethanol, sections were immersed in Bouin's Solution overnight at room temperature. Sections were incubated in working Weigert's Iron Hematoxylin Solution to stain nuclei, Biebrich Scarlet-Acid Fuchsin to identify the cytoplasm and muscle fibers, and Aniline Blue Solution to stain collagen fibers. Whole heart images were taken with the Nikon Eclipse Ti2 microscope.
   Cytokine Release Assays: The Proteome Profiler Mouse Cytokine Array A, (ARY006, R&D Systems) was used to quantify the 40 mouse proteins (cytokines, chemokines, and growth factors) from serum collected the day before surgery, and four hours after the 2^{nd} injection from mice injected with PBS, unmodified MSCs, and ZipperCells. Serum was diluted and mixed with a cocktail of biotinylated detection antibodies, according to the manufacturer's instructions. The sample/antibody mixture was then incubated with the array membrane overnight at 4° C. The membranes were washed and incubated with streptavidin-horseradish peroxidase followed by chemiluminescent detection. The array data were quantitated to generate a protein profile and the results are presented as the average signal (pixel density) of the pairs of duplicate spots representing each cytokine or chemokine analyzed using MATLAB. The data presented are from three biological samples per group.

Detection of Antibody Responses (ELISA): Mice were injected with B-LZ & C-LZ 10 nM ZipperCells, or PBS, as previously described. After 6 weeks, blood was collected. The serum was obtained by centrifugation of blood for 20 minutes at 1500×g. Nunc MaxiSorp plates were coated with B-LZ or C-LZ 10 nM at 1 μg/ml and incubated overnight at 4° C. The plates were thoroughly washed 3× with 0.1% PBS-Tween (PBST), and incubated with ELISA blocking buffer (3% (w/v) instant dry milk (Food Lion) in PBS) for 1 h at RT. The wells were again washed 3× with 0.1% PBST. Serum was added to the top wells at a 1:100 dilution in ELISA blocking buffer and then serially diluted 3-fold followed by a 2 h incubation at RT. Plates were washed again 3× with 0.1% PBST. Washes were followed by the addition of 100 μL of Goat Anti-Mouse IgG Fc-HRP (Cat #1033-05, SouthernBiotech) for 1 h at room temperature. The wells were washed 5× with 0.1% PBST followed by the addition of the TMB-ELISA substrate. After a 10 minute incubation, 50 μl of 2 N H2SO4 was added to stop the reaction. The absorbance was measured at 450 nm and 570 nm with a SpectraMax M5 plate reader (Molecular Devices, Sunnyvale, CA).

Data analysis and statistics: Comparisons between groups were analyzed using one-way ANOVA with a post hoc Tukey test (*p<0.05, **p<0.01, ***p<0.001). All statistical analyses were performed using Prism 7 (GraphPad Software, La Jolla, CA). The error bars represent standard deviations (SD).

### Example 1

### Design and Characterization of the ZipperCell Depot System

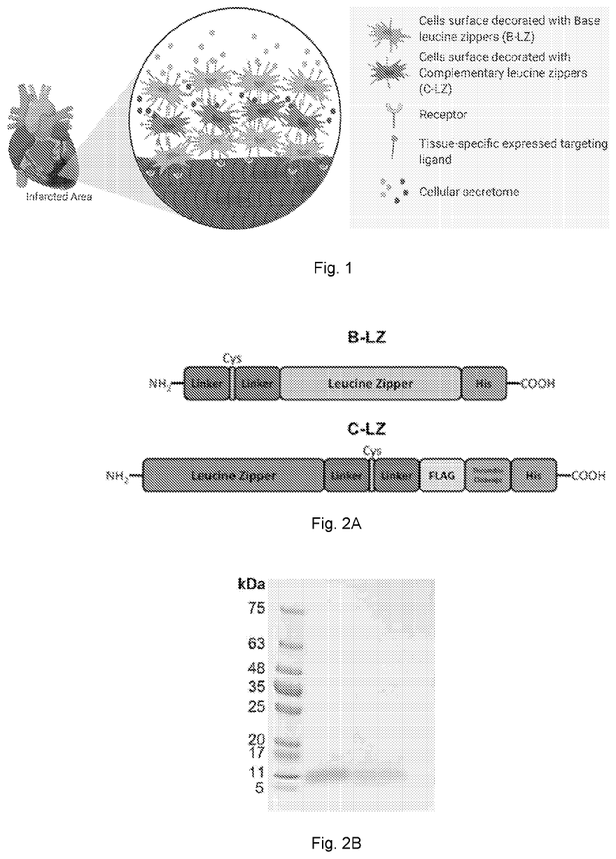

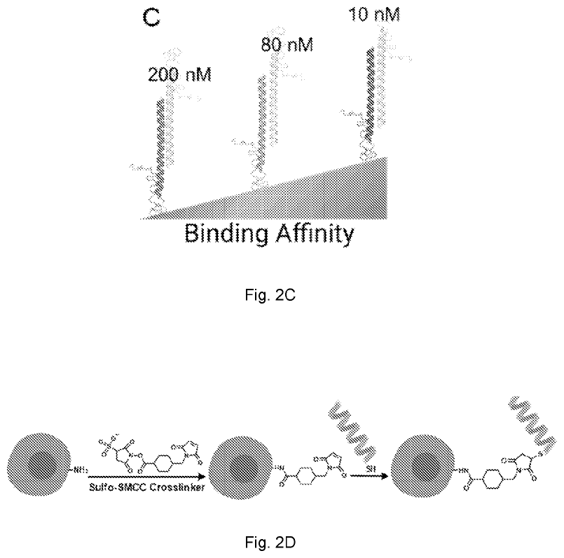

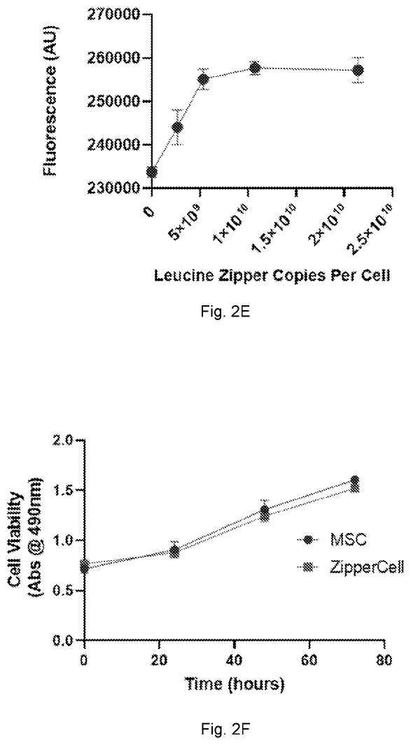

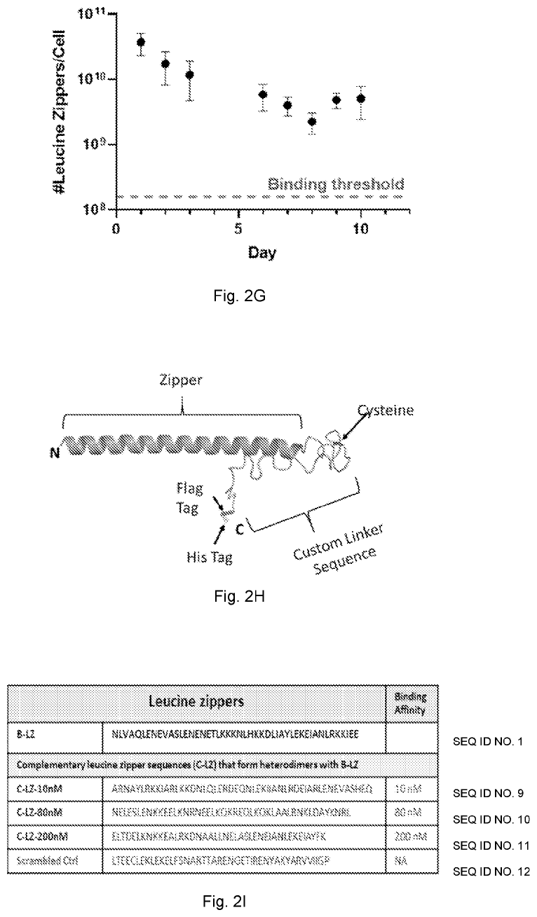

The ZipperCell depot system centers, in some embodiments, around the use of a library of heterodimerizing, synthetic leucine zippers for physical crosslinking mediated by competition-based dimerization. In vivo, sequential administration of leucine zipper decorated cells facilitates the physical crosslinking and therefore retention and accumulation of cells at the target site, including for example the infarct site (FIG. 1). Leucine zippers were generated from SynZip sequences which have been further customized to include a Gly-Ser linker chain, a single cysteine for site-specific thiol maleimide conjugation, and a polyhistidine tag for purification (FIGS. 2A & 2G). These customized constructs can be expressed and purified with high yield in an E. coli system (FIG. 2B). The leucine zipper set includes 3 pairs of leucine zippers with varying affinities (for example, but not limited to, about 10, 80, and 200 nM) and scramble control (FIGS. 2C & 2I).

Once expressed, leucine zippers were conjugated to the surface of mesenchymal stem cells. First, a heterobifunctional crosslinker, such as but not limited to sulfosuccinimidyl 4-(N-maleimidomethyl)cyclohexane-1-carboxylate (Sulfo SMCC), was covalently bonded to amines of the cell surface. Next, thiol-containing leucine zippers were conjugated to the crosslinker (FIG. 2D). By varying concentrations of fluorescently labeled leucine zippers, the saturating dose of leucine zipper and control leucine zipper density on the cell surface was determined (FIG. 2E). It was then determined, via MTS assay, that ZipperCell surface decoration did not significantly affect cell viability (FIG. 2F). Next, computational modeling was used to predict the number of leucine zippers required per cell to allow for stable network formation under in vivo blood flow conditions. By accounting for factors such as flow rate, blood pressure, leucine zipper length, and affinity, it was determined the minimum number of leucine zippers required per cell. For zipper dimerization at 200, 80, and 10 nM binding affinity (Kd), the number of zippers required was about 2.8×10⁸, 2.4×10⁸, and 2.2×10⁸/cell, respectively (FIG. 2G). To verify that the extent of the disclosed leucine zipper decoration surpassed this calculated threshold and could be maintained as the cells expanded, resulting in ligand dilution, leucine zipper expression on cells was tracked over time in vitro. It was found that even after 10 days, leucine zippers were surprisingly still present at about 10 fold the required density (FIG. 2G).

### Example 2

### Verification of Leucine Zippers Heterodimerization

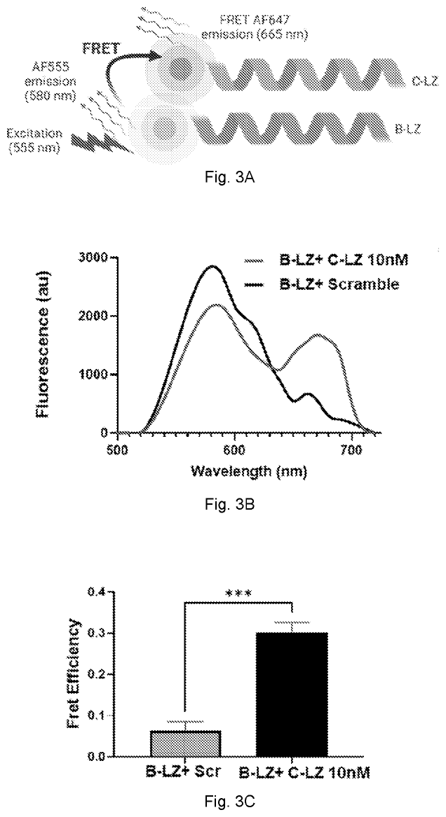

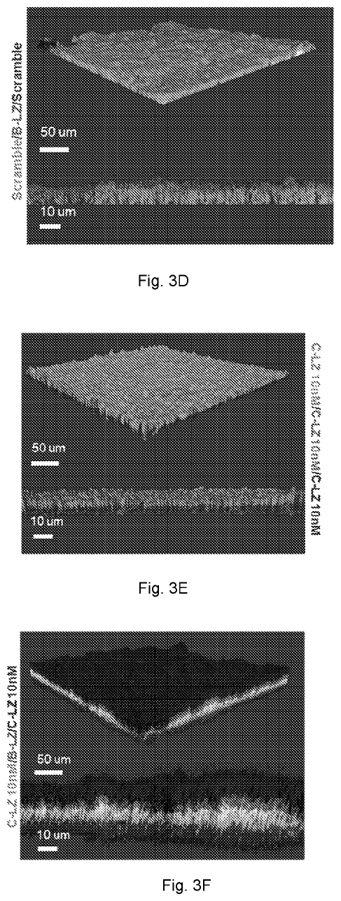

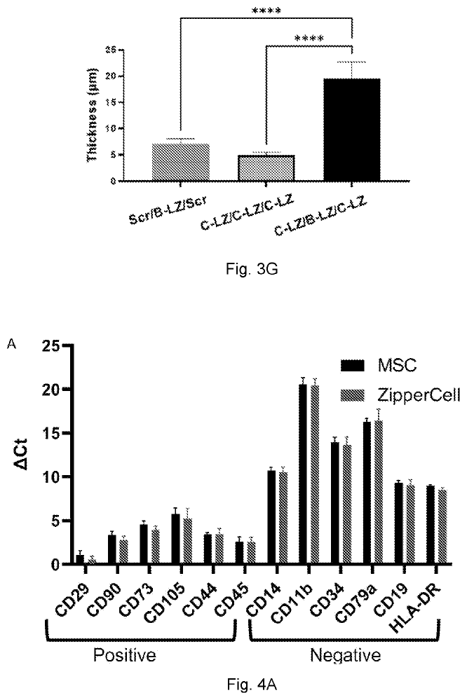

To verify that the disclosed custom-modified synthetic leucine zippers could heterodimerize, FRET assays were utilized (FIG. 3A-C). When dimerized in the expected parallel orientation, C-terminally labeled AF555 B-LZ leucine zippers transfer energy to cysteine labeled AF647 C-LZ leucine zippers. This resulted in a greater FRET efficiency than mixtures of B-LZ with scrambled control sequences, pointing to the specificity of heterodimerization in the selected pairs. Finally, it was assessed whether the presence of heterodimerizing leucine zippers on the surface of cells could facilitate the physical crosslinking of specific cells to form cell sheets. It was observed that when base leucine (B-LZ) ZipperCells were seeded in a monolayer, only complementary (C-LZ) ZipperCells bound, resulting in multilayered cell layer sheets with increased thickness (FIG. 3F). However, cultures with scrambled control or only a single leucine zipper were unable to dimerize and were unable to form multi-layered cell sheets (FIG. 3D-E). Quantification of layer thickness confirmed that the successful layer formation resulted in a three-fold greater thickness compared with only a cell monolayer from the scrambled (Scr) protein (Scr Cells/B-LZ Cells/Scr Cells) or only the C-LZ 10 nM protein (C-LZ Cells/C-LZ Cells/C-LZ Cells) (FIG. 3G).

### Example 3

Confirmation that ZipperCells Maintain Normal Phenotype

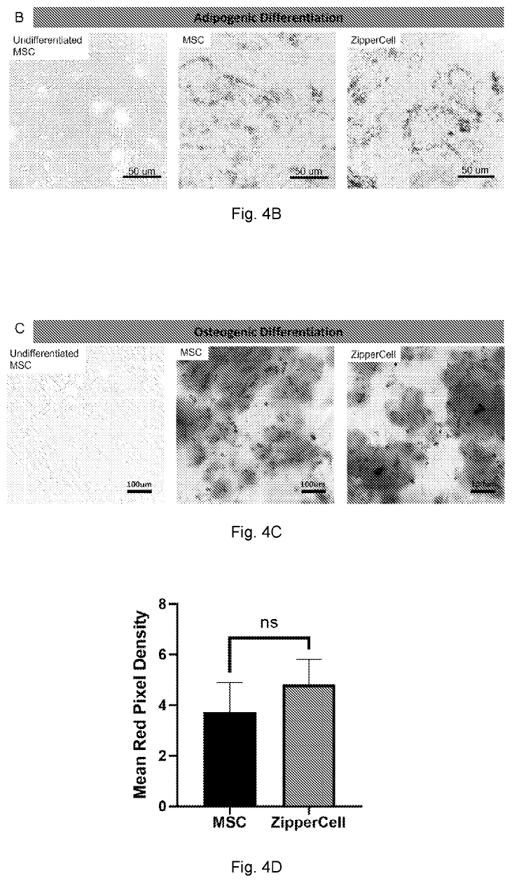

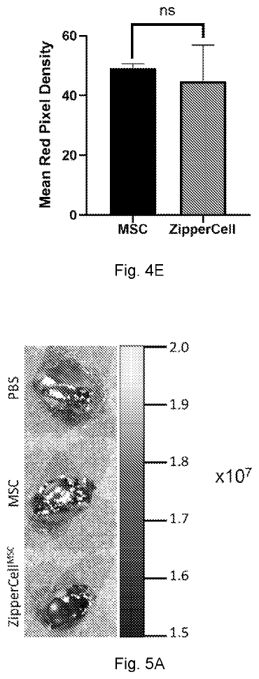

A functional concern of the ZipperCell system was whether they would maintain their “stemness” upon surface decoration. To assess the MSC phenotype, RT PCR was performed. A panel of positive phenotypic MSC markers such as CD29 & CD90 and negative phenotypic markers (CD14 & CD19) was tested, using GAPDH as a housekeeping gene. I was found that all genes were consistent. Upon further investigation, it was also found that the ZipperCells maintained their capacity to differentiate into osteoblasts and adipocytes. This indicated their ability to retain their phenotype associated with regenerative wound healing, which is needed at the target site for treatment or therapy, including for example at the infarct site. No statistical difference was found between ZipperCells and unmodified MSCs 7 days after surface decoration when examining relative fold change in gene expression (FIG. 4A). Additionally, upon adipogenic and osteogenic differentiation induction, both unmodified MSCs and ZipperCells were able to maintain their capacity to differentiate with no statistical difference in Alizarin Red or Oil Red staining (FIG. 4B-E).

### Example 4

Leucine Zipper Decorated Cells Accumulate after Ischemia/Reperfusion

After the characterization of ZipperCell crosslinking in vitro, the cardiac accumulation of the ZipperCell system in vivo was assessed. To begin with, the biodistribution of DiR-labeled stem cells after systemic administration via tail vein injection was examined in a mouse model of myocardial infarction (MI) (ischemia/reperfusion).

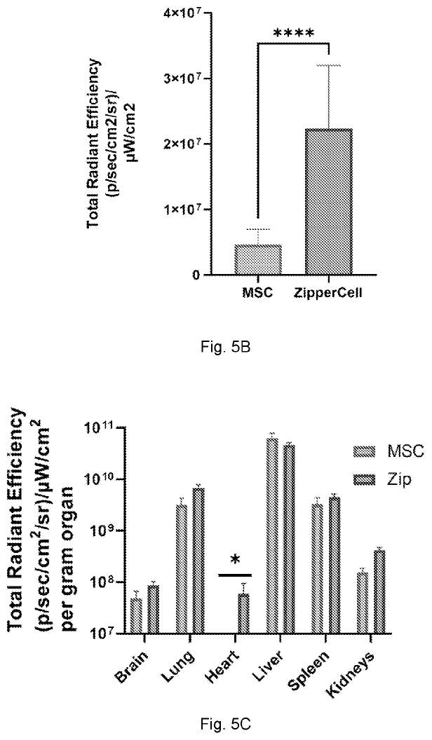

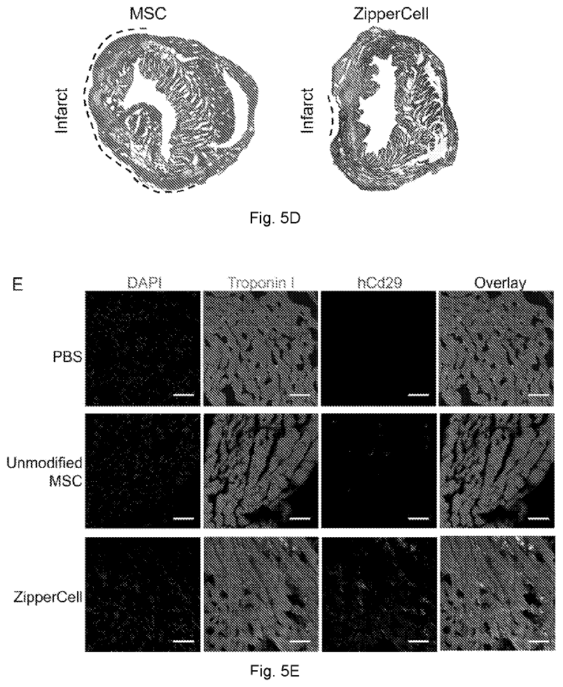

Twenty-four hours after surgery, 500,000 cells were injected into mice every 12 hours for a total of three injections. Twelve hours after the final injection, organs were collected, and their DiR fluorescence was imaged ex vivo, using the IVIS Spectrum in vivo imaging system. Although the majority of both unmodified MSCs and ZipperCells were found in the liver, spleen, and lungs (FIG. 5C), quantification of the fluorescence in the hearts surprisingly revealed 486±126% higher levels of ZipperCells compared to unmodified MSCs 72 hours post-infarction (FIGS. 5A & 5B). Representative sections were taken within the border zone and infarct area, which was defined as the visible infarct area and the 2 mm area encircling the infarct area (FIG. 5D)^{[34,35]}. Upon immunohistochemical staining, it was found that 9.97±0.24-fold greater deposition of ZipperCells within the infarct and border zone of hearts compared to unmodified MSCs (FIG. 5E), likely as a result of leaky vasculature and enhanced permeability after MI.

### Example 5

### Long-Term Retention of ZipperCells

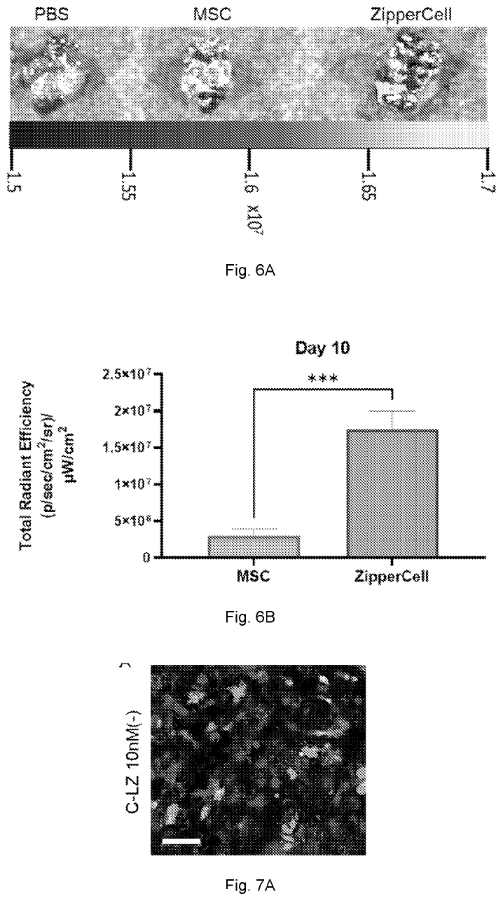

After demonstrating that almost 500% more ZipperCells were retained at the infarct site 72 hours after ischemia/reperfusion when compared to unmodified MSCs, efforts were undertaken to investigate the potential for retention at longer time points. It was found that 10 days after the first injection, 78±11% of the 72-hour ZipperCell population fluorescent signal was still detectable in the mouse hearts via ex vivo IVIS imaging, an about 6-fold improvement over unmodified MSCs (FIG. 6A-B). This suggests that the fold retention of ZipperCells over unmodified MSCs increases over time.

### Example 6

### Competitive Leucine Zippers Facilitate Targeted Disassembly

In some embodiments it can be advantageous to have the “disassembly-on-demand” feature when using multi-dose crosslinking, to ensure personalized clinical safety by design. Competitive ligand binding was used to test if the scaffold and cell clusters can be disassembled.

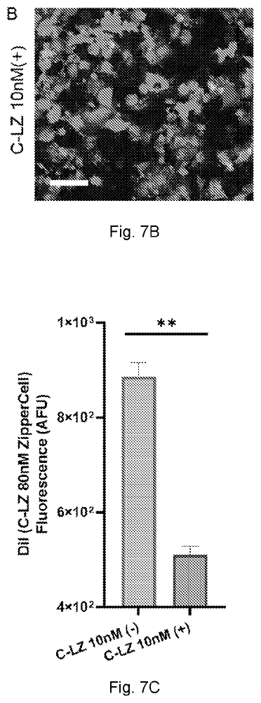

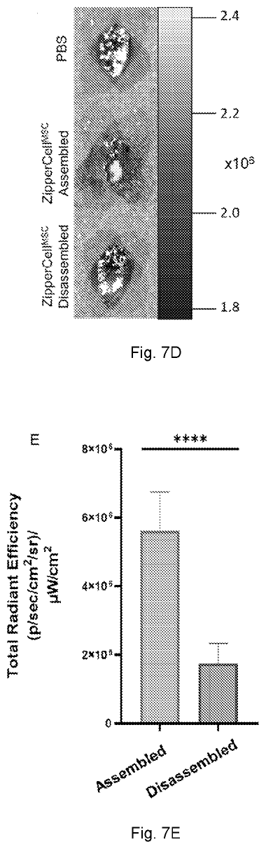

It was hypothesized that lower affinity leucine zipper pairs can be knocked off by dosing with a competing high-affinity leucine zipper, leading to disassembly of the system. This built-in safety mechanism may allow for rapid removal in the case of potential toxicity or immunogenicity. To test this in vitro, a monolayer of B-LZ leucine zipper ZipperCells stained with DiO (green) was seeded. Then DiI (red) fluorescently labeled populations of C-LZ 80 nM affinity ZipperCells were independently incubated (FIG. 7A). Following a 3-hour incubation, wells were then treated with 100× fold excess of free C-LZ 10 nM peptide for an additional 3 hours (FIG. 7B). It was observed that after C-LZ 10 nM incubation effectively removed the majority of the C-LZ 80 nM signal (FIG. 7C). Finally, to determine the in vivo effects of competition-mediated disassembly, the C-LZ 80 nM affinity leucine zipper pair was dosed based on the previously described schedule. Twenty-four hours after the final injection, each mouse was dosed with one mg of free C-LZ 10 nM. Twelve hours later, mice were euthanized, and their organs were imaged (FIG. 7D). A 68.8±10% decrease in the DiR signal at the infarct of mice treated with the highest affinity leucine zipper (FIG. 7E) was observed.

### Example 7

### ZipperCells are Non-Immunogenic and Maintain Cytokine Expression Levels

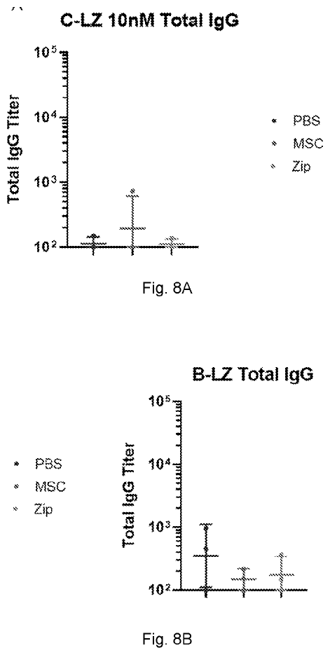

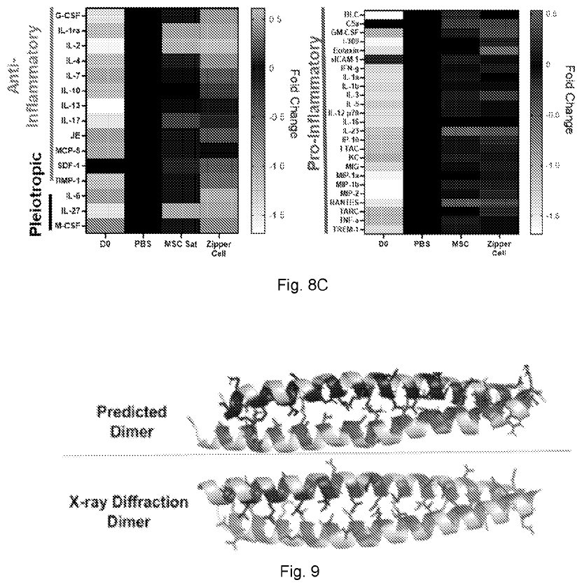

Next, to verify that the long-term accumulation and retention of ZipperCells would not cause toxicity, anti-drug antibody assays were performed. ELISA assays using either C-LZ 10 nM (FIG. 8A) or B-LZ (FIG. 8B) as the coated antigen, were used to detect total IgG content 6 weeks after injection. Very low antibody titers were found in all injected mice, signaling a lack of immune response. As additional mediators of systemic inflammatory responses, the impact of multiple ZipperCells administrations on cytokine profiles were also investigated. To complete this, serum samples were collected from mice the day before surgery (D0) and 4 hours after the second injection (40 hours post-MI). Serum levels of 40 cytokines were then tested using the Proteome Profiler Cytokine Array, Panel A. It was observed that induction of MI overall increased cytokine expression in all mice, including the PBS control. Both groups of cell-treated mice showed similar profiles of pro-inflammatory cytokines, but ZipperCell treated mice mediated robust upregulation of several increased anti-inflammatory and pleiotropic cytokines (FIG. 8C), likely due to enhanced accumulation. After the second dose, a statistically significant increase was found in multiple anti-inflammatory factors such as G-CSF, IL-4, IL-6, and SDF-1 in ZipperCell treated mice compared to unmodified MSC-treated mice, which have been shown to reprogram recruited macrophages to an anti-inflammatory phenotype^{[36-38]}. Overall, this data supports a biocompatible delivery system.

### Example 8

### Discussion of Examples 1-7

Mesenchymal stem cells (MSCs) are exemplary mediators of wound healing. Their ability to self-renew, immunomodulate, and differentiate into a variety of cell types makes them ideal tools for regenerative medicine, and their ability to reduce infarct size, reduce fibrosis, and promote new vascularization at the wound site makes them particularly suited for treating myocardial infarction. Despite these successes, there are still no FDA-approved MSC treatments and an overwhelming consensus within the field is that poor accumulation and retention are primary hurdles to clinical translation.

Disclosed herein is an engineered system of self-assembling MSCs with multiple flexible features and fine-tune ability for enhanced accumulation and retention. An essential component of the system is the layer-by-layer dosing strategy. The disclosed approach allows several waves of cellular delivery in a non-invasive manner through circulation. Unlike conventional delivery strategies, which do not allow the subsequent accumulation of cells or carriers after saturation of the target infarct site, each dose of cells in the disclosed platform serves as an additional capturing surface for the next dose of cells. This dramatically amplifies the targetable surface area for additional waves of cell attachment. Additionally, the extensive multivalent crosslinking helps resist venous washout, allowing cells to persist long term.

While unmodified MSCs showed only 0.15±0.07% retention of the injected dose at the infarct site, which is in line with previously published results, ZipperCells showed about 500% improvement at the infarct site compared to unmodified MSCs. This improved retention is further widened by day 10, where ZipperCells demonstrate a about 600% improvement over unmodified MSCs.

Another key feature of the ZipperCell system is its non-invasive, yet targeted, systemic administration. MSCs in the ZipperCell system migrate to the site of infarct and promote cardiac repair. Without being bound by any particular theory or mechanism of action, MSCs expressing chemokine receptors, including CXCR4, can migrate to the site of injury via chemoattraction. This is shown by the preferential retention in the ischemic myocardium region by both unmodified MSCs and ZipperCells.

In clinical settings of MI, it is standard practice to establish intravenous access in at least one peripheral vein and maintain this access for the duration of the hospital stay to administer any IV therapies. As such, ZipperCell administration would be painless and unobtrusive for the patient. The average length of hospital stay after MI in the United States is 3 days and longer in other countries, so ZipperCell infusion is not expected to prolong the stay. Moreover, several clinical studies have shown that patients tolerate multiple MSC injections with no adverse effects.

Recent strategies for enhancing cellular retention have largely focused on shielding cells from venous washout. Encapsulating materials, such as injectable hydrogels, are of great interest as they provide protective structural support and can be comprised of a range of materials. Scaffolds made of materials such as injectable fibrin hydrogels or polymer fibers have garnered particular preclinical interest. While these materials may be engineered to incorporate additional therapeutic elements and tunability, this also requires extensive material characterization of factors such as stiffness, porosity, and adhesion. Furthermore, they are often plagued by low cellular or payload capacity. The inclusion of additional materials widens the likelihood of toxicity and immunogenicity as well as the alteration of cellular function and engraftment in vivo. Other approaches such as cell sheets and cardiac patches attempt to overcome poor retention through the delivery of cells with established extracellular matrices. Recently, a four-dimensional GelMA patch was engineered to account for biomechanical and physicochemical properties, in which cell engraftment was significantly improved. While strategies like this have advanced the field with insight into biomimetic design, they are also bound by extensive microfabrication equipment costs and lengthy culture protocols. In contrast, the disclosed simplified approach circumvents these challenges and allows ZipperCells to form their own scaffold-like network in situ, making them more potent and biocompatible.

A unique component of the ZipperCell system is the intrinsic safety-by-design feature. Unlike traditional therapeutic depots, ZipperCell disassembly or removal can be performed without surgery, which could cause additional acute stress to the patient and increase the possibility of morbidity. The disclosed disassembly mechanism uses a simple intravenous injection to remove the majority of all cells at the target site. The ability to rapidly and non-invasively remove the therapeutic depot is especially important in the case that patients begin to show adverse effects or toxicity and need to terminate the treatment immediately. Furthermore, conventional therapeutic depots such as the aforementioned hydrogels and patches cannot be easily modified, making it difficult to adjust drug regimens in response to the evolving disease state, which is becoming ever more important to optimize outcomes in the personalized medical era. While the disclosed results show that ZipperCells are well-tolerated and do not induce significant immune responses, this mechanism adds to the overall potential clinical translation of the disclosed system.

While the presently disclosed subject matter exploits the fact that mesenchymal stem cells are naturally able to migrate to sites of inflammation, it is expected that in some embodiments the incorporation of a targeting ligand on the cell surface would likely further enhance accumulation at the site. The use of alternative therapeutic carriers such as nanoparticles, microparticles, and mixed systems is also provided which further underscore the wide utility of this newly engineered system. Furthermore, dosing intervals can be optimized as needed.

### Example 9

### Conclusions from Examples 1-8

Disclosed herein is the first-of-its-kind, non-invasive, self-assembling cell depot for significantly enhanced retention and accumulation at the infarct site. This novel cell delivery strategy amplifies the available binding area in a layer-by-layer fashion. Additionally, these systems and methods ensure minimal invasiveness for not only initial delivery but also for removal, as needed. As disclosed herein, the cell surface decoration of leucine zippers does not interfere with the MSC phenotype or the capacity to differentiate can facilitate cellular crosslinking to retain cells at the infarct site effectively. Conceptually and technically, this platform can serve as a proof-of-concept for a variety of non-invasive, in situ forming depots. The present disclosure also enhances the understanding of the biocompatibility and immunogenicity of leucine zipper-based therapeutics in combination with stem cell therapy.

### Example 10

### Novel Synthetic Heterodimerizing Leucine Zippers

The novel synthetic heterodimerizing leucine zippers are applicable to any of the applications and examples discussed hereinabove.

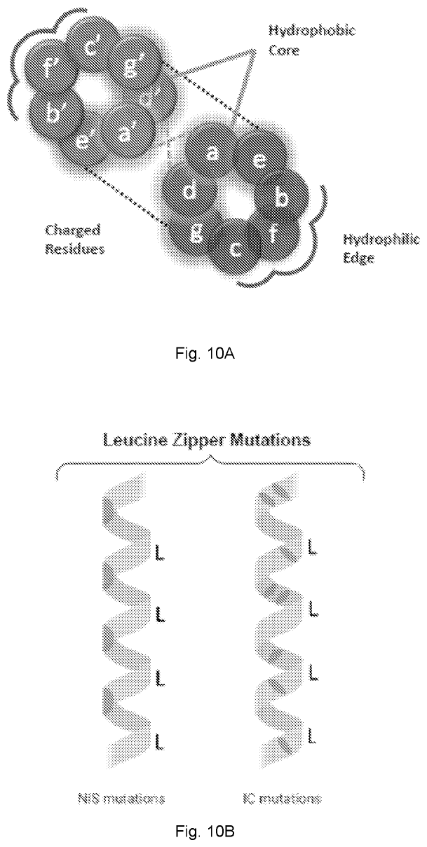

FIG. 9 shows a comparison between the disclosed molecular docking prediction of an established synthetic heterodimerizing leucine zipper pair. Intermolecular interacting residues are bolded (both in the dimer illustrations in the top panel as well as in the side-by-side amino acid nomenclatures in the bottom panel (note the amino acid nomenclatures in the bottom panel are not shown for the sequence per se but instead for comparison purposes to demonstrate predictability). This shows that the disclosed molecular docking procedure leads to reliable predictions in binding affinity between leucine zippers.

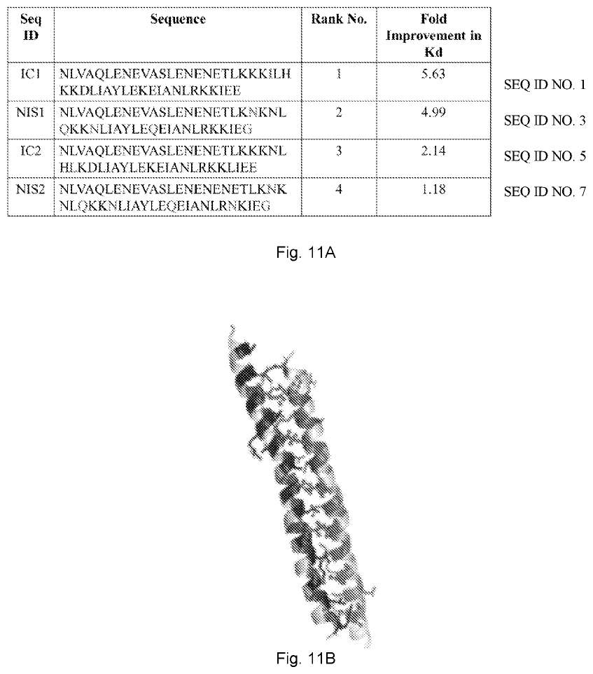

FIGS. 10A and 10B shows an illustrative representation of optimal leucine zipper amino acid properties. By mutating amino acids (FIG. 10B) to enhance hydrophilicity of positions c, f or b, it is possible, as demonstrated herein, to alter the Non-Interacting Surface (NIS) of the leucine zipper chain. By mutating amino acids to increase the charged and hydrophobic content within positions e, g or a, d, respectively, it is possible to alter the interfacial contacts (IC) and therefore the strength of interactions between leucine zipper chains (FIGS. 10A and 10B).

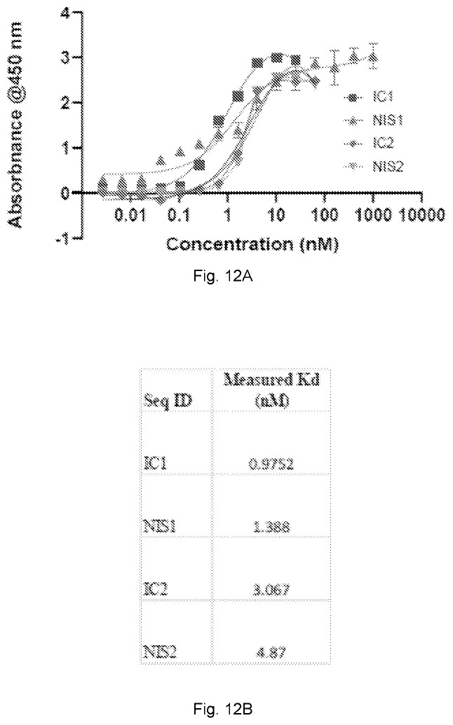

FIG. 11 shows a listing of select leucine zipper mutant protein sequences with mutated amino acids shown in red/highlights (FIG. 11A). By incorporating these mutations, the disclosed molecular docking predictions show enhancement in the number of intermolecular interacting residues (FIG. 11B; shown by different shades of branching and darked chain components).

Below are the sequences (amino acid and nucleotide sequences) for each of the leucine zippers of FIG. 11A:

IC1 Protein/amino acid (SEQ ID NO. 1):
NLVAQLENEVASLENENETLKKKILHKKDLIAYLEKEIANLRKKIEE
IC1 DNA/nucleotide (SEQ ID NO. 2):
AACTTAGTTGCTCAACTAGAGAATGAAGTAGCGAGCCTGGAAAACGA
GAACGAAACCCTGAAAAAGAAGATCTTGCACAAAAAGGACCTGATTG
CATACTTGGAGAAAGAGATCGCTAATCTGCGTAAAAAGATCGAGGAA
NIS1 Protein/amino acid (SEQ ID NO. 3):
NLVAQLENEVASLENENETLKNKNLQKKNLIAYLEQEIANLRKKIEG
NIS1 DNA/nucleotide (SEQ ID NO. 4):
AACCTTGTGGCCCAATTAGAGAACGAGGTGGCCTCCCTGGAAAATGA
GAACGAAACATTAAAAAACAAAAACTTGCAGAAGAAAAACTTAATCG
CCTACTTGGAACAAGAGATTGCTAACTTACGCAAGAAGATTGAAGGG
IC2 Protein/amino acid (SEQ ID NO. 5):
NLVAQLENEVASLENENETLKKKNLHLKDLIAYLEKEIANLRKKIEE
IC2 DNA/nucleotide (SEQ ID NO. 6):
AACTTAGTTGCTCAACTAGAGAATGAAGTAGCGAGCCTGGAAAACGA
GAACGAAACCCTGAAAAAGAAGAACTTGCACCTAAAGGACCTGATTG
CATACTTGGAGAAAGAGATCGCTAATCTGCGTAAAAAGATCGAGGAA
NIS2 Protein/amino acid (SEQ ID NO. 7):
NLVAQLENEVASLENENETLKNKNLQKKNLIAYLEQEIANLRNKIEG
NIS2 DNA/nucleotide (SEQ ID NO. 8):
AACCTTGTGGCCCAATTAGAGAACGAGGTGGCCTCCCTGGAAAATGA
GAACGAAACATTAAAAAACAAAAACTTGCAGAAGAAAAACTTAATCG
CCTACTTGGAACAAGAGATTGCTAACTTACGCAACAAGATTGAAGGG

FIG. 12 shows binding curves (FIG. 12A) used to determine relative binding affinities (FIG. 12B) of the top four mutated protein sequences to a previously established leucine zipper sequence (SynZip2). These show that the disclosed modeling and subsequent mutational incorporation produces functional proteins with enhanced binding affinity. Such enhanced binding affinities were not expected or predictable.

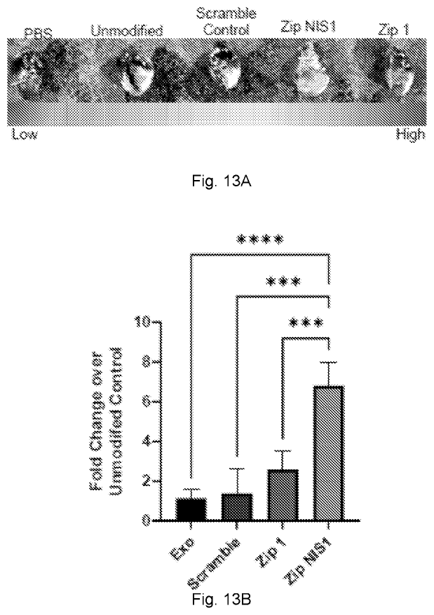

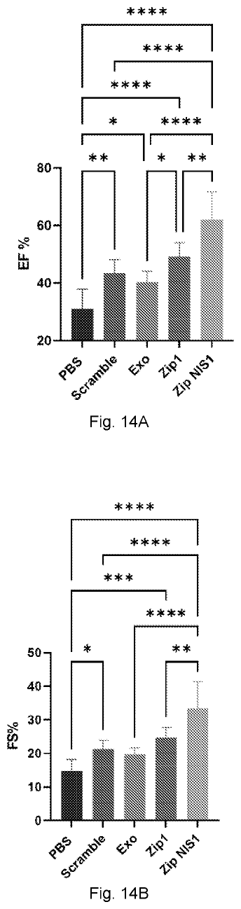

FIG. 13 shows the cardiac accumulation of fluorescently labeled nanosized exosomes (Exo) injected via tail vein into a mouse model of myocardial infarction. Myocardial infarction was induced in C57BL/6 mice (female, 10 to 12 weeks of age) via a left lateral thoracotomy to expose the heart, and a permanent ligation of the left coronary artery (LCA) (FIG. 13A). Ex vivo IVIS imaging of 3 alternating doses of DiIC₁₈(7) (1,1′-Dioctadecyl-3,3,3′,3′-Tetramethylindotricarbocyanine lodide (DiR) labeled exosomes surface decorated with heterodimerizing leucine zippers, 48 hours after the first injection (60 hours post-MI). Exosomes, also known as small extracellular vesicles (EV), were isolated via differential centrifugation from murine, bone-marrow derived mesenchymal stem cells. A scramble control containing an identical amino acid content without a leucine heptad was included as a control. (FIG. 13B) This shows that the disclosed novel leucine zipper sequences, specifically (but not limited to) NIS1 facilitate about 7-fold greater in vivo accumulation when compared to unmodified exosome control. Data are mean±sd. **P<0.01 by one way ANOVA. N=4.

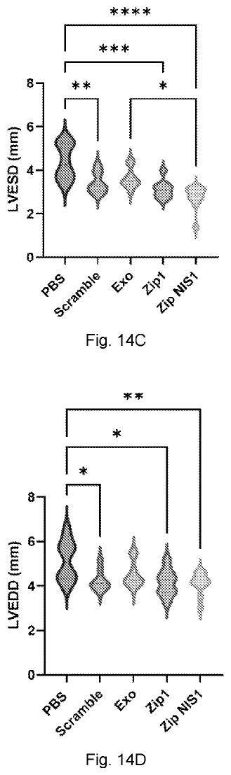

FIG. 14 shows echocardiographic parameters of cardiac function determined via parasternal short axis view. This shows that mesenchymal stem cell derived exosomes surface decorated with the disclosed novel protein sequences facilitate a highly statistical improvement in cardiac function by measures of ejection fraction (FIG. 14A; EF), fractional shortening (FIG. 14B; FS), left ventricle end systolic diameter (FIG. 14C; LVESD) and left ventricle end diastolic diameter (FIG. 14D; LVEDD). Data are mean±sd. **P<0.01 by one way ANOVA. N=8-10

### Materials and Methods for Examples 11-18

Molecular docking & in silico binding. Three-dimensional structural models of all leucine zipper proteins were generated using ITASSER based on the known structure of homologous proteins. Energy-minimized models of base leucine zippers (B-LZ) and complementary leucine zippers (C-LZs) were molecularly docked using HADDOCK to generate dimers. Amino acids designated within the “A, D, E, and G” heptad positions were marked as active residues, and a single model was chosen as the final conformation. Binding affinity and intermolecular interactions (Kd) were determined using PRODIGY.

Protein expression & purification. All leucine zipper sequences were cloned into a pET21a vector (Novagen, Burlington, MA) and expressed in BL-21 (DE3) cells (Lucigen, Middleton, WI). Bacterial cultures were grown overnight at 37° C. in 2× YT (Fisher Scientific, Waltham, MA) medium and ampicillin. For large-scale production, a 10 L autoinduction^{[31]} culture was inoculated with 1% of the overnight culture and grown for four hours at 37° C. and then an additional 20 hours at room temperature. After incubation, cells were harvested 16 h post-induction by centrifugation. The cell pellet was resuspended in lysis buffer containing 50 mM Tris, 100 mM KCl, 10% glycerol, 100 μg/ml lysozyme, and 1 mM Phenylmethanesulfonylfluoride Fluoride (PMSF), pH 8.0. The cell suspension was sonicated and then centrifuged at 17,000×g for 40 minutes at 4° C., after which Ni-NTA Resin (G-Biosciences, St. Louis, MO) was added to the solution. The beads were incubated with the solution and the protein was isolated by immobilized metal affinity chromatography (IMAC)^{[32-34]}. Endotoxin removal was performed using Pierce High-Capacity Endotoxin Removal resin (Thermofisher) and validated using the Pierce LAL Chromogenic Endotoxin Kit Quantitation Kit (Thermofisher).

Enzyme-linked immunosorbent assay (ELISA). Nunc MaxiSorp 96-well plates were coated with Base-Leucine Zipper (B-LZ) at 2.5 μg/mL and incubated overnight at 4° C. The plate was thoroughly washed with 0.1% TBS-Tween (TBST). The wells were washed with 0.1% PBST and then blocked with BSA blocking buffer for 1 h at room temperature before incubation with complementary leucine zipper (C-LZ) concentrations ranging from 1 μM to 0.3 μM at room temperature for 2 hours before washing. Washes were followed by the addition of 100 μL of anti-FLAG horseradish peroxidase (HRP)-linked monoclonal antibody (1:10,000 mAb, #A8592, Sigma Aldrich, St. Louis, MO) for 1 h at room temperature with rocking. The wells were washed with 0.1% PBST followed by the addition of TMB-ELISA substrate. After a 10 min incubation, 2 M H₂SO₄ was added to stop the reaction. The absorbance was measured at 450 nm and 570 nm with a SpectraMax M5 plate reader (Molecular Devices, Sunnyvale, CA).

Cell culture and EV isolation. Conditioned medium was collected from murine bone-marrow-derived mesenchymal stem cells (Cyagen MUBMX-01101) grown to 70% confluency and then incubated with EV-depleted FBS-medium for 48 hours. EVs were isolated by differential centrifugation according to previously established protocols.

Protein-EV conjugation. EV concentration was determined by nanoparticle tracking analysis (NTA). At a concentration of 1.0e10 particles/ml, cells were incubated with 2.5 μM DiR [DiIC18(7) (1,1′-dioctadecyl-3,3,3′,3′-tetramethylindotricarbocyanine iodide] dye and incubated with 500 μg sulfosuccinimidyl 4-(N-maleimidomethyl)cyclohexane-1-carboxylate (Sulfo-SMCC) for 30 minutes at 37° C. To reduce protein for conjugation, proteins were incubated with 1 mM tris(2-carboxyethyl) phosphine (TCEP) at room temperature for 20 minutes. After washing EVs with Amicon Ultra-15 100 kDa MWCO centrifugal filter units, 2 mg of protein was added to EVs and incubated at room temperature for 30 minutes before final washing and resuspension.

EV characterization. EVs were prepared as previously described, and their concentration and size were evaluated by NTA (Particle Metrix, ZetaView). For transmission electron microscopy (TEM), isolated EVs were incubated with glow discharged Formvar/carbon-supported copper TEM grids. Samples were allowed to adsorb on the grid for 2 min before wicking off with a filter paper. 1% uranyl acetate was applied to the grid for staining, and the excess was wicked off immediately and repeated again. Finally, grids were allowed to air dry and then imaged using the FEI Tecnai T12 TEM at 120 V.

Enzyme-linked immunosorbent assay (ELISA). Nunc MaxiSorp 96-well plates were coated with leucine zippers at 2.5 μg/mL and incubated overnight at 4° C. The plate was thoroughly washed with 0.1% TBS-Tween (TBST). Wells were washed with 0.1% PBST and then blocked with BSA blocking buffer for 1 h at room temperature before incubation with a stock of 3.3 μM C-LZ at 37° C. for 2 hours before washing. Washes were followed by the addition of 100 μL of anti-FLAG horseradish peroxidase (HRP)-linked monoclonal antibody (mAb, #A8592, Sigma Aldrich, St. Louis, MO) for 1 h at room temperature with rocking. The wells were washed with 0.1% PBST followed by the addition of TMB-ELISA substrate. After 10 minutes of incubation, 2 M H₂SO₄ was added to stop the reaction. The absorbance was measured at 450 nm and 570 nm with a SpectraMax M5 plate reader (Molecular Devices, Sunnyvale, CA).

Patterned EV binding. Individual glass microscope slide sections were circled with a hydrophobic pen and were coated with poly-D-lysine (0.01% solution) for 1 hour at room temperature. After washing three times with deionized water, the slides were wrapped tightly in a single layer of parafilm. Patterns were cut into the parafilm using a 30-gauge needle and then incubated with B-LZ (2.5 mg/ml) solution for three, 1 h incubations. The parafilm was removed and the outlined regions were blocked with 5% milk solution for 1 hr. Sections were then incubated with DiI (DiIC18(3); 1,1′-dioctadecyl-3,3,3′,3′-tetramethylindocarbocyanine) labeled unmodified EVs or Zippersomes at a concentration of 1e14 particles/ml for three hours, followed by washing, and mounting with Fluorsave (Millipore, Burlington, MA). Images were taken with the Nikon Eclipse Ti2 microscope.

Cellular EV Uptake. Unmodified EVs and Zippersomes, were labeled with DiI (1,10-dioctadecyl-3,3,30,30-tetramethylindocarbocyanine perchlorate). RAW 264.7 macrophages, human umbilical vein endothelial cells (HUVECs) (ATCC, Cat #CRL-1730), HL-1 cardiomyocytes (Millipore, Cat #SCC065), and human cardiac fibroblasts (HCFs) (Sciencell, Carlsbad, CA; Cat #6300) cells were used as representative cell types within the infarct microenvironment. Cells were seeded at a density of 5,000 cells/well in 96 well plates and left to adhere overnight. HUVECs, HL-1s, and HCFs were maintained in hypoxic conditions (5% CO2, 5% 02) for 24 hours prior to studies. After 24 h, conditioned media (CM) from all cell types were collected, mixed, and dosed to RAW 264.7 cells to mimic infarct microenvironment exposure. The remaining cells were given fresh medium. Cells were treated with doses of 1.11e8 EVs/well each hour, followed by washing for a total of three doses within the first three hours. Eight hours after the first dose, wells were washed and fixed with 4% paraformaldehyde before imaging with a Nikon Eclipse Ti2 microscope.

Animals. Female 10-12-week-old Female C57BL/6 mice were used for the in vivo studies. The University of North Carolina at Chapel Hill Institutional Animal Care and Use Committee (IACUC) approved all animal procedures. All methods and experiments were performed in accordance with the U.S. National Institutes of Health Guide for Care and Use of Laboratory Animals. Humane care and treatment of animals were ensured.

Mouse model of MI (permanent coronary ligation). All surgeries were performed in the McAllister Heart Institute (MHI) Cardiovascular Physiology and Phenotyping Core. Briefly, mice were anesthetized with isoflurane. An incision was made to visualize the trachea before intubating with a 20-gauge blunt needle and ventilation. A left lateral thoracotomy exposed the heart, and the left coronary artery (LCA) was identified and permanently occluded with a 7-0 nylon suture. Occlusion was confirmed by electrocardiography (ECG) and visual inspection of myocardial pallor. The thorax was closed in layers (ribs, muscles, and skin). Mice were provided with analgesics and monitored per protocol.

Biodistribution of EVs. For biodistribution studies, 1.3e9 DiR-labeled Zippersomes or unmodified MSC derived EVs, or PBS was administered via tail vein injection into C57BL/6 mice 24 hours after inducing MI. A total of three injections were administered every twelve hours. Twelve hours after the final injection (60 hours post-MI or 21 days post-MI), mice were sacrificed and the brain, lung, heart, liver, spleen, and kidneys were collected and weighed. Fluorescent biodistribution was analyzed using the IVIS Spectrum in vivo imaging system (PerkinElmer, Waltham, MA). Average region of interest (ROI) signals were calculated using Aura Imaging Software (Spectral Instruments, Tucson, AZ). The data is presented as total radiant efficiency/g organ.

Histopathological assessment. For histolopathological studies, 1.3e9 DiR-labeled Zippersomes or unmodified MSC EVs, or PBS were administered via tail vein injection into C57BL/6 mice. A total of three injections were administered every twelve hours.
(1) Assessment of infarct size: 21 days post-MI, mice (n=8-10) were briefly perfused with KCl (30 mM) to arrest the heart in diastole, sacrificed, and hearts collected. Harvested hearts were fixed in formalin for two days, dehydrated, and then cleared before paraffin embedding. Tissues were then cut into 7.5 μm sections, which were deparaffinized and rehydrated for subsequent staining. Masson's trichrome staining was performed using a kit (Sigma-Aldrich, Burlington, MA). Whole heart images were taken with the Nikon Eclipse Ti2 microscope. Midline left ventricle lengths were measured using ImageJ software.
(2) Histological assessment of CD45 signal in livers: After day 21 sacrifice, livers were frozen and embedded in optimal cutting temperature (OCT) compound. Tissues (n=3) were cut as 8 μm sections and immediately fixed in ethanol. Sections were then incubated with anti-mouse CD45 (Tonbo Biosciences, San Diego, CA; Cat #70-0451-U100) at a 1:200 dilution for 30 minutes at room temperature. This was followed by an HRP secondary antibody (Tonbo Biosciences, San Diego, CA, Cat #72-8104-M001). Chromogen was developed with a DAB HRP substrate (Vector Labs, Newark, CA; Cat #SK-4105). Samples were then counterstained with hematoxylin and bluing reagent (Biovision, Milpitas, CA) before mounting with Permount. Images were taken with the Nikon Eclipse Ti2 microscope.

Echocardiography. Echocardiograms were collected with the Vevo2100 Ultrasound system (VisualSonics, Toronto, Canada). Two-dimensional M mode parasternal short axis views were recorded. Left ventricular dimensions, wall thickness, heart rate, and cardiac output were recorded. Left ventricle (LV) stroke volume (SV) was calculated as the difference between the end diastolic volume (EDV) and the end systolic volume (ESV). Ejection fraction (EF) was calculated as (EF=[(EDVESV)/EDV]*100).

Spatial analysis. Serial sections from histological tissue assessment were utilized for fluorescence staining (7.5 μm cleared paraffin-embedded sections, prepared as previously described). Sections were stained with a panel of 12 fluorescently conjugated antibodies. Next sections were cover slipped and mounted with Fluorsave (Millipore) and imaged using mosaic tile imaging on an Olympus FV3000RS confocal microscope.

For image analysis, tiles were exposed to flatfield correction (BioVoxxel, ImageJ) and then deconvolved and stitched using Huygens software (Scientific Volume). Stitched files were imported into Imaris, and downstream analysis was performed as outlined in the CytoMap Spatial Analysis methods.

Statistical analysis. All the quantitative data were presented as mean±SD. The mean values are based on a minimum of n=3 biological replicates. Groups were compared using one-way ANOVA with a post hoc Tukey test (*p<0.05, **p<0.01, ***p<0.001). All statistical analyses were performed using Prism 7 (GraphPad Software, La Jolla, CA). The error bars represent standard deviations (SD).

### Example 11

### High-Affinity Leucine Zippers

To enhance the accumulation and retention of intravenously-injected EVs for the treatment of MI, an additional approach to the disclosed ZipperCell platform with small nanosized EVs was developed. By utilizing heterodimerizing leucine zippers and decorating them on vesicle surfaces, the present disclosure facilitates vehicle crosslinking, in situ. To further improve potential accumulation in vivo, a library of mutated versions of a previously tested complementary leucine zipper was created, C-LZ 10 nM to increase binding affinity to the base leucine zipper (B-LZ). By taking the hydrophobic, hydrophilic, and electrostatic interactions that reinforce and stabilize leucine zipper dimers into account (FIG. 10A), a group of mutations for this study was carefully selected; three non-interacting surface (NIS) mutations, three interfacial contact (IC) mutations, and three sequences that were a combination of NIS and IC mutations (FIG. 10B). The binding affinities of these nine sequences were first determined through molecular docking simulations and then rank ordered based on their binding strengths.

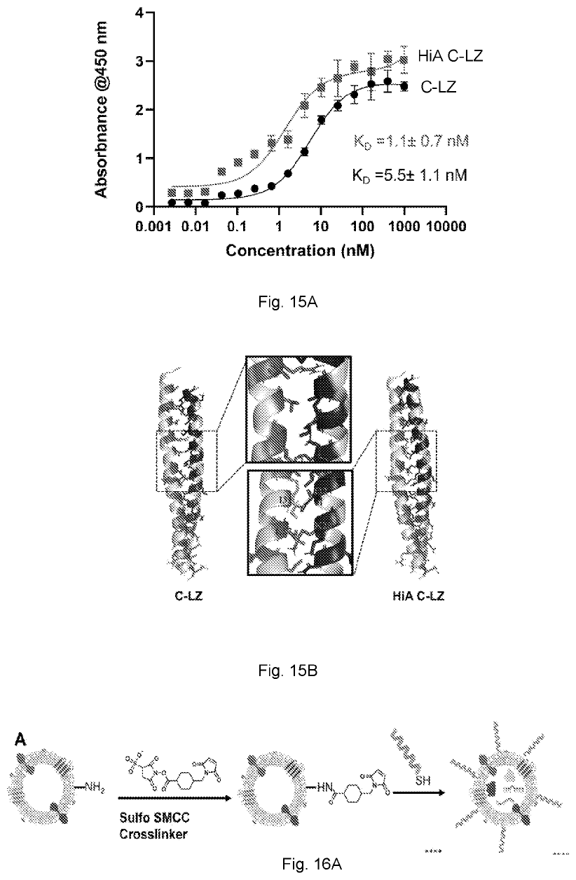

The proteins were then expressed for in vitro testing. Using titration ELISA, the binding affinity of each sequence was tested for its affinity to the base leucine zipper (B-LZ). Upon reviewing the top four candidates (FIG. 11A), NIS1 was selected, hereby termed high-affinity complementary leucine zipper (HiA C-LZ), for downstream studies (although the remaining novel leucine zippers disclosed herein are expected to be equally effective). This decision was based on its high binding affinity and high expression levels in E. coli. In particular, this protein demonstrated a five-fold improvement over B-LZ binding compared with previously tested C-LZ 10 nM (FIG. 15A). The in silico results were consistent with this finding, showing a significant increase in intermolecular interactions and binding affinity between NIS1 and B-LZ as compared to C-LZ10 nM and B-LZ (FIG. 15B).

### Example 12

### Characterization of MSC Zippersomes

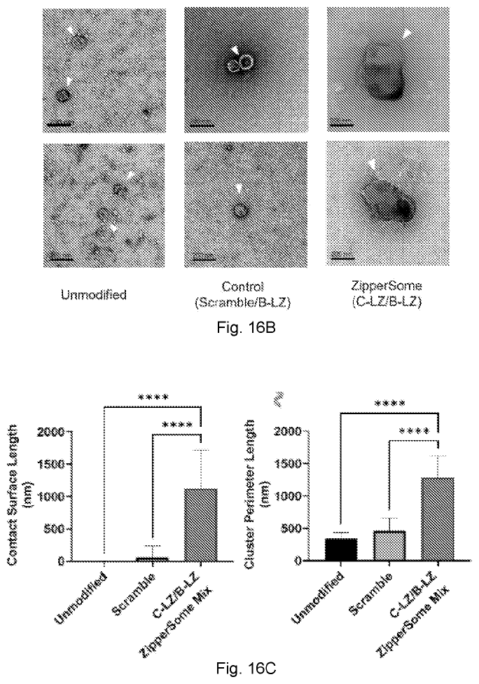

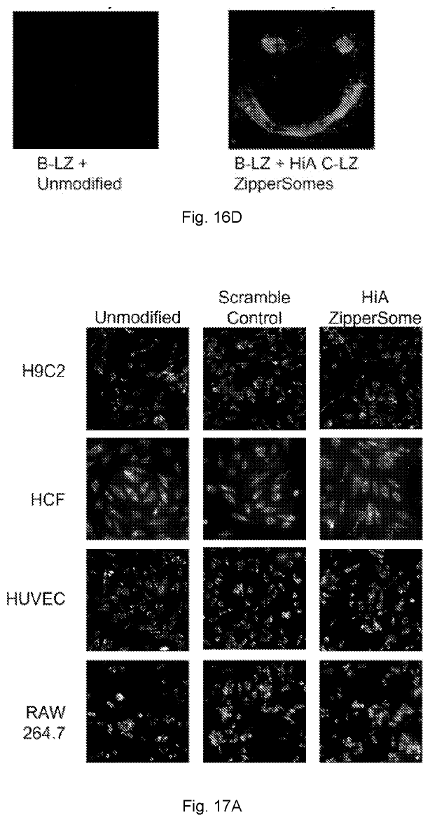

After optimizing leucine zipper binding affinity, the HiA C-LZ was attached to the surface of EVs to create Zippersomes. This was achieved with a heterobifunctional crosslinker (other suitable cross-linkers disclosed herein), Sulfo SMCC, where the amines on the EV surface were conjugated to the free thiols of either the B-LZ or a C-LZ (FIG. 16A). To investigate whether the improved leucine zipper binding affinity translated into increased retention and accumulation, new sequences were tested. To validate that this property could be extended to smaller vehicles, within the nanometer range, EVs were also decorated with B-LZ and C-LZ proteins to create Zippersomes. This was further validated with TEM, where unmodified EVs and scramble control Zippersomes were present as individual vesicles whereas B-LZ/C-LZ Zippersomes clustered (FIG. 16B) with extensive contact area (FIG. 16D). The Zippersomes showed a 1124.23±594.4 nm increase in contact surface length compared with unmodified EVs and exhibited about 4-fold and about 1.5-fold increases in perimeter length compared with unmodified EVs and EVs surface-decorated with a scrambled control peptide, again providing evidence of leucine zipper-mediated crosslinking. Lastly, to further validate the hetero-specificity of C-LZ Zippersomes, a “smiley face” pattern of B-LZ protein was plated and blocked, and the pattern only appeared when fluorescently labeled HiA Zippersomes were incubated (FIG. 16C). Without modification, fluorescently-labeled EVs were unable to bind and no detectable signal was observed (FIG. 16C).

### Example 13

### Zippersomes Exhibit Enhanced Cellular Binding and Uptake In Vitro

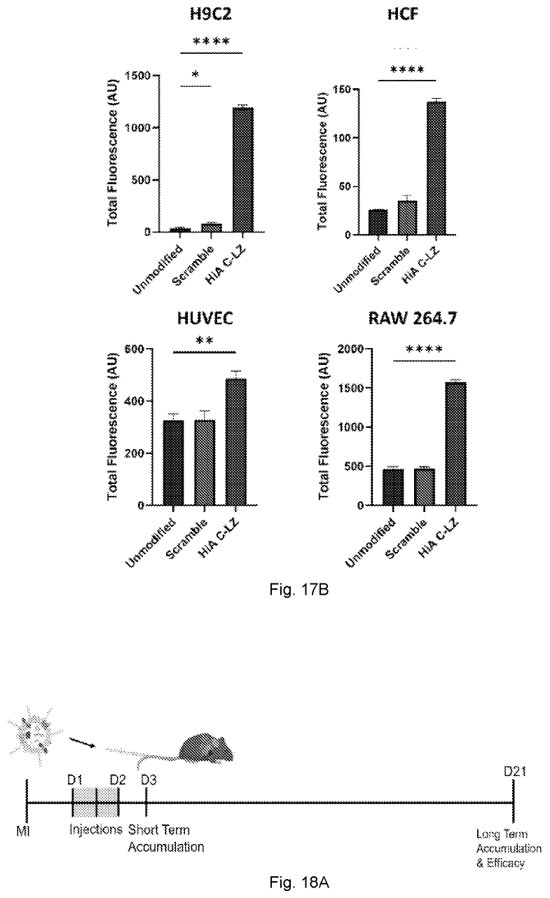

Experiments were conducted to determine if Zippersome crosslinking would enhance cellular interactions within the infarct microenvironment. To assess cellular interactions in vitro, four representative cell types [rat ventricular cardiomyoblasts (H9c2s), fibroblasts (HCFs), endothelial cells (HUVECs), and macrophages (RAW 264.7)] were tested for EV uptake. Cardiomyoblasts, fibroblasts, and endothelial cells were preconditioned in hypoxia chambers prior to EV incubation, while macrophages were treated with the hypoxia-conditioned medium (CM) along with the dosed EVs, all to effectively mimic the hypoxic infarct site (FIG. 17A). After three doses of alternating Zippersomes, it was observed that for all cell types, there was significantly more HiA Zippersome uptake compared with unmodified EVs or scramble control Zippersomes (FIG. 17B). Additionally, H9C2 cells and macrophages bound and took up the most Zippersomes. Collectively, these results demonstrate unexpected and superior cellular binding and uptake of Zippersomes compared with unmodified EVs.

### Example 14

### Zippersome Accumulation and Retention In Vivo

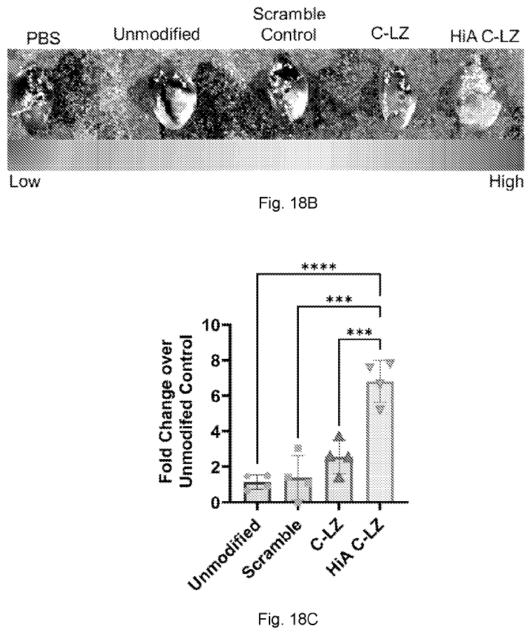

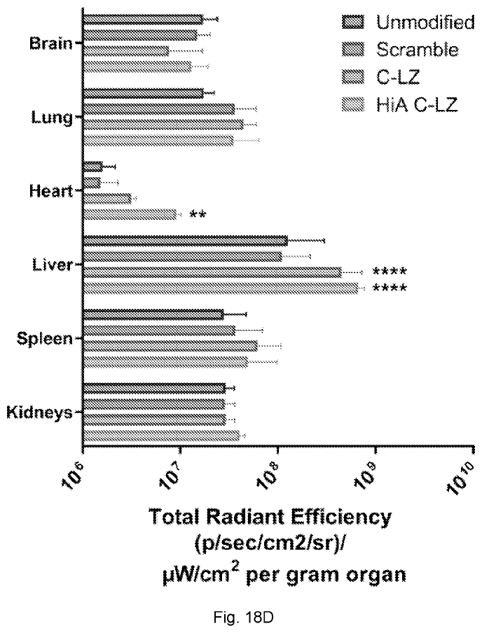

Next, the biodistribution of DiR-labeled MSC Zippersomes after intravenous administration was assessed. EVs and Zippersomes were administered as previously described: 24 hours after MI, three doses of complementary EVs were administered sequentially at 12 h intervals (FIG. 18A). Importantly, 60 hours post-MI, mice treated with C-LZ and HiA Zippersomes exhibited 2.5 and about 7-fold greater retention than the unmodified EV control, respectively (FIG. 18B-C). These results show significantly improved accumulation and retention, specifically at the infarct site. Interestingly, there were also slightly increased fluorescence levels in mice dosed with heterospecific Zippersomes (C-LZ & HiA C-LZ) 12 hours after the final and third injection (FIG. 18D-E). Overall, these findings demonstrate significant shifts in biodistribution to the infarcted myocardium due to in situ leucine zipper crosslinking and persistence of the disclosed therapeutic vesicles.

### Example 15

### Zippersomes Persist in the Infarcted Heart for Up to 21 Days

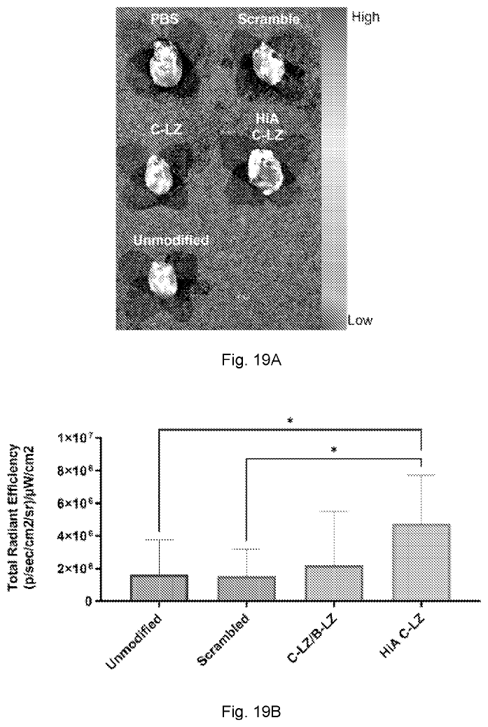

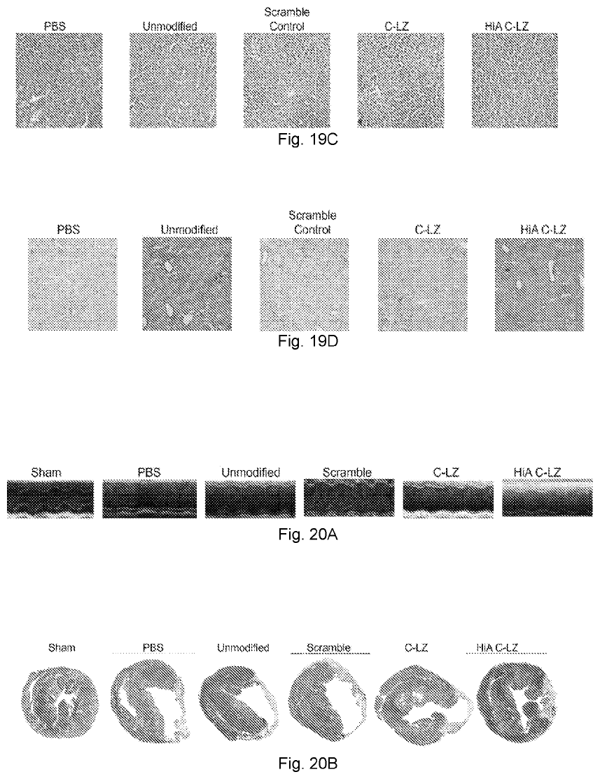

It was next determined whether the crosslinking mediated by the leucine zippers also prolonged retention of Zippersomes in the infarcted myocardium. Surprisingly, it was found that there were still detectable levels of DiR signal from HiA Zippersomes-treated mice within the infarcted myocardium after 21 days (FIG. 19A). This translated into significantly more fluorescent EV signal compared with unmodified EVs and the scramble control (FIG. 19B). This is the longest reported retention of intravenously injected EVs in the infarcted myocardium.

To evaluate the biocompatibility of the Zippersomes, histopathological assessment of liver tissue on day 21 after Zippersome administration was performed, with anti-CD45 antibodies used to identify immune cell infiltrates. There was no significant signal within any of the group, and the tissues were histopathologically normal (FIG. 19 D-E). These findings collectively indicate a lack of unfavorable immune responses or tissue damage in the liver.

### Example 16

### Zippersome Treatment Improves Cardiac Function

It was next determined whether the in vivo effect of intravenously injected Zippersomes on fibrosis and cardiac function after MI using a mouse model of permanent coronary artery ligation. Cardiac contractile function was tested by echocardiography in six experimental groups: sham, MI+PBS, MI+unmodified EV, MI+scramble control, MI+Zippersomes, MI+C-LZ Zippersomes, and MI+HiA Zippersomes (FIG. 20A). The same dosing schedule of three injections at 12 h intervals was utilized. After 21 days, HiA Zippersome-treated mice showed significantly improved cardiac function (FIG. 20C). HiA Zippersome-treated mice displayed significantly higher fractional shortening (FS) (33.4±8.0% vs 19.6±2.0%) compared with mice treated with unmodified EVs. Additionally, systolic LV diameter was also substantially decreased (2.8±0.6 mm vs 3.6±0.8 mm), consistent with improved contractile function (FIG. 20C). Finally, HiA Zippersome mouse hearts had significantly less fibrosis (midline length %) (20.1±7.2% vs 43.2±6.7%) compared with unmodified EVs (FIG. 20B). Overall, these findings demonstrate a marked improvement in multiple cardiac functional parameters after MI after HiA Zippersome treatment.

### Example 17

### Zippersome Treatment Reduces Fibrosis and Immune Cell Infiltration in Spatial Analyses

Finally, experiments were conducted to examine and quantify the infiltration of immune cells such as macrophages, T cells, and B cells as well as resident myocardial endothelial cells and fibroblasts, with the expectation that enumerating these cell populations would provide key insights into the remodeling processes occurring within Zippersome-treated hearts. To determine the spatial residencies of these model cell types, multiplex fluorescent staining and spatial analysis, paired with deconvolution, were performed. Imaris Surface creation and CytoMap software were used for further analysis.

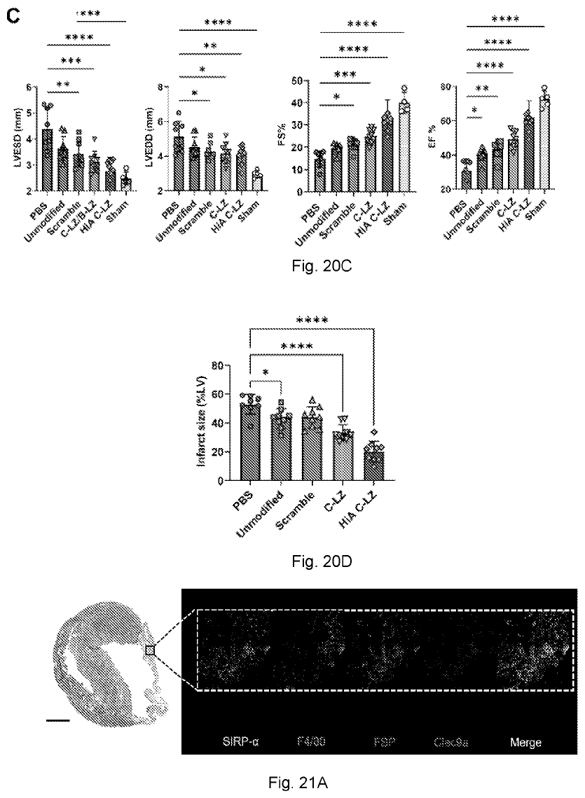

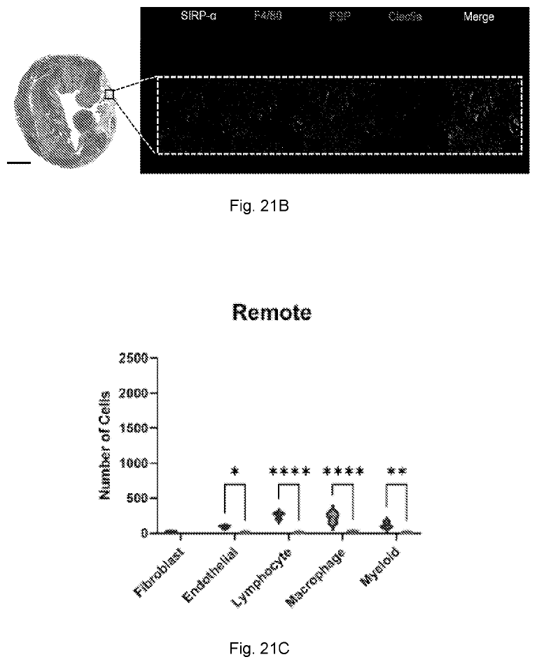

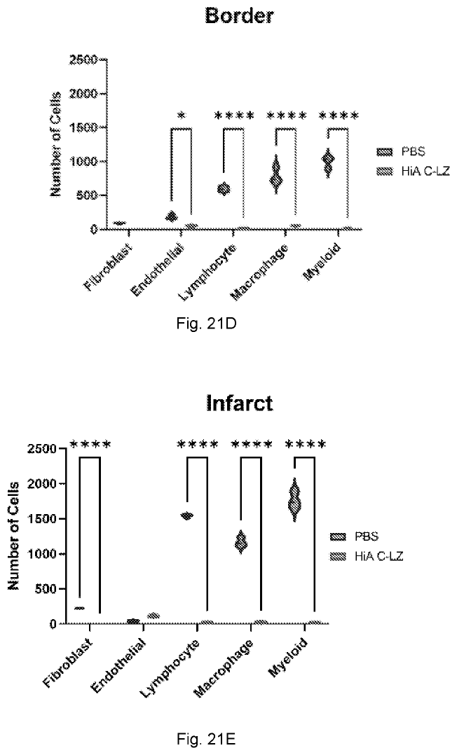

Multiplex spatial staining captured highly complex cell populations throughout the samples (FIG. 21A-B). Representative fluorescence images are shown. There were significantly denser populations of detected myeloid, lymphocyte, macrophage, and fibroblast cells within PBS-treated control mouse hearts than in HiA Zippersome treated mice. These populations, especially fibroblasts, were largely located within the infarct region and border zones corresponding to areas of fibrosis, suggesting injury-specific cell recruitment. Additionally, there was a significant mixture of cell types within regions of the PBS samples, whereas HiA Zippersome hearts displayed distinct regions with defined niches. Furthermore, within PBS samples, most cells were located within the border and infarct zones, while HiA Zippersome hearts had significantly less cell infiltration and a more even distribution between areas (FIG. 21C-E).

Lastly, endothelial cells (CD31 expression) and their relative location were assessed. The percentage of endothelial cells in PBS samples was significantly smaller than those in HiA C-LZ samples. Furthermore, endothelial cells in the PBS samples were largely localized in the border and remote areas, whereas endothelial cell signals were present in all zones of HiA Zipersome samples, including the infarct zone (FIG. 21C-E). Further inspection revealed that within PBS mice, the majority (71.7±10.8%) of identified macrophages were the proinflammatory M1 phenotype (F4/80⁺Cd11b⁺Cd11c⁺), while only a small proportion (16.9±3.3%) of macrophages identified within HiA Zippersome hearts were M1 type. Overall, these data provide insights into the primary cell types present within the injury site and potential mechanisms of action.

### Example 18

### Discussion of Examples 11-17

MSC-derived EVs have been used as a promising alternative to live cell therapies for the treatment of MI and wound healing. Many studies have now shown that EVs are key mediators of stem cell-mediated wound healing and regeneration within the damaged heart, largely due to their ability to move, even through occluded sites. However, despite their widespread use within the field, they are often implemented via direct myocardial injection, which poses risks and does not overcome limited retention. In addition to the inherent risk and limited translatability of this invasive administration route, rapid biodistribution and clearance of these vehicles is often observed, so the therapeutic effects are often limited at the target site. The Zippersomes introduced in this disclosure provide new approaches and mechanisms to address these hurdles.

Here it was aimed to reduce invasive procedures, improve cellular uptake, and accumulate EVs at the infarct site for an extended period to improve therapeutic efficacy and cardiac function long-term. Testing was conducted to determine if leucine zipper crosslinking could also be used to enhance the accumulation and retention of EVs to increase cellular uptake and therefore improve cardiac function. To facilitate crosslinking of EVs, heterodimerizing leucine zippers were attached to two sets of EVs, which were subsequently dosed in an alternating manner. The data show that the leucine zippers are biocompatible and can stably bind long term within serum conditions to facilitate cellular crosslinking in vivo.

The cellular uptake of EVs is important to consider, since their circulatory half-life is typically very brief (t_{1/2}<30 min). By increasing the number of EVs clustering at the cell surface, cellular uptake, or internalization mediated by endocytosis, phagocytosis, or membrane fusion, tests were conducted to determine whether overall cellular uptake could be enhanced. Increased cellular uptake of EVs is particularly important within inflammatory disease sites, such as infarct sites, where EV persistence is necessary to regulate inflammatory responses. First, it was found that when complementary Zippersome populations are mixed, they can crosslink and form large, defined clusters. Furthermore, it was found that crosslinking and cluster formation increased cellular binding and uptake, especially within cardiomyocytes and macrophages. Previous studies have shown that MSC-derived EVs possess anti-inflammatory effects and are able to polarize inflammatory macrophages present in the infarcted myocardium after MI, towards an anti-inflammatory phenotype, decreasing the number of inflammatory M1 macrophages, suggesting a possible mechanism of action for EV-mediated regeneration in vivo. This mechanism is supported by the presently disclosed in vivo studies, where it was found that within the PBS-treated control mice, greater than 70% of macrophages were the pro-inflammatory M1 phenotype (F4/80+Cd11b+Cd11c+), while the number of M1 macrophages identified within HiA Zippersome hearts substantially decreased to about 16.9%.

Unmodified EVs are typically cleared within hours of systemic administration. Encouragingly, it was discovered here that through the disclosed vehicle surface decoration, it can increase cardiac retention of MSC EVs up to day 21. This level of retention, particularly through systemic administration, has never been demonstrated before (published studies typically report retention of up to 24 hours maximum). Although slightly enhanced retention of Zippersomes within livers was observed compared with unmodified EVs, signs of toxicity or immune infiltrates within these livers was not observed.

Additionally, it was discovered that Zippersomes, especially those containing the high binding affinity leucine zipper (HiA C-LZ), enhanced the therapeutic effect of MSC EVs and substantially improved cardiac function, likely due to increased retention at the target site. On day three, an about 7-fold increase in Zippersome accumulation was observed within the heart compared with unmodified EVs. On day 21, it was found that cardiac contractile function substantially increased while infarct size and LV diameter significantly decreased compared with both PBS- and unmodified EV-treated mice. Notably, the Zippersomes were able to restore cardiac function of mice back to healthy levels. While most other EV MI studies have shown modest improvements in fibrotis in the range of approximately 10-20% compared with vehicle-treated control animals, the data disclosed herein show an impressive 32.7% improvement in infarct size reduction. Collectively, these findings clearly demonstrate that retention can be controlled by altering the leucine zipper binding affinity.

Upon spatial cell analysis, PBS samples showed extensive fibroblast and immune cell residency within sites of injury, consistent with well-characterized post-infarct pathobiology. The presence of dense mixtures of lymphocytes and myeloid cells, interspersed with pockets of fibroblasts, was detected. Previous studies have shown that myeloid cells and lymphocytes infiltrate ischemic and infarcted areas, where they work in tandem as part of the collective immune response to remodel the myocardium.

Within the HiA Zippersome-treated mice, there was a substantial reduction in all inflammatory cell types, including macrophages, likely indicating a reduced inflammatory environment resulting in reduced fibrosis.

One major advantage of the disclosed compositions, formulations and systems is its high level of tunability. For example, Zippersomes can be combined with various therapeutic cargoes and drug carriers, including for example, but not limited to, small molecules, nucleic acids (non-coding and coding), and proteins. Additionally, Zippersomes can be applied to different disease states where enhanced retention can provide therapeutic benefits where targetable surface areas are limited. Because each therapeutic dose is designed to capture the next therapeutic dose, the present disclosure provides a highly effective strategy to increase drug accumulation and retention. Intravenous injection was selected as the route of administration due to its clinical translatability. Typically, patients diagnosed with MI receive continuous IV infusions throughout their hospital stay, making it a convenient and accessible route for multiple dosing strategies.

### Conclusions from Examples 11-18

In conclusion, through surface engineering, leucine zippers can be used to generate EVs capable of amplifying the targetable surface areas and crosslinking in situ. Sequence-specific crosslinking improves cellular uptake for more potent therapeutic mechanisms. Intravenously administered Zippersomes accumulate at the site of MI and are retained for several weeks. Long-term retention significantly improved cardiac function, as shown by a remarkable decrease in fibrosis, and an improvement in ejection fraction and fractional shortening. These novel formulations and methods provide a significant improvement in EV delivery and other drug delivery carriers.

It will be understood that various details of the presently disclosed subject matter may be changed without departing from the scope of the presently disclosed subject matter. Furthermore, the foregoing description is for the purpose of illustration only, and not for the purpose of limitation.

1. A non-invasive, in situ forming depot for delivery of a therapeutic agent, comprising:
one or more heterodimerizing, synthetic leucine zippers for physical crosslinking mediated by competition-based dimerization; and
a therapeutic agent,
wherein the one or more heterodimerizing, synthetic leucine zippers form a self-assembling depot of the therapeutic agent at a target site in vivo.

2. The non-invasive, in situ forming depot for delivery of a therapeutic agent of claim 1, wherein the heterodimerizing, synthetic leucine zippers amplify an available binding area at a target site in vivo in a layer-by-layer fashion.

3. The non-invasive, in situ forming depot for delivery of a therapeutic agent of claim 1, wherein the therapeutic agent comprises an affinity or attraction to a target site in vivo, optionally comprising a targeting ligand on a surface of the therapeutic agent, to enhance accumulation of the therapeutic agent at the target site.

4. The non-invasive, in situ forming depot for delivery of a therapeutic agent of claim 1, wherein the non-invasive, in situ forming depots for delivery of a therapeutic agent achieve significantly enhanced retention and accumulation of the therapeutic agent at the target site, optionally wherein the non-invasive, in situ forming depots, including the therapeutic agent, are configured to persist at the target site for at least about 5 days, about 10 days, about 15 days, about 20 days, about 30 days, about 50 days, about 100 days, or more.

5. The non-invasive, in situ forming depot for delivery of a therapeutic agent of claim 1, wherein the therapeutic agent comprises a cell, optionally a stem cell, a vesicle, an extracellular vesicle (EV), a nanoparticle, a microparticle, a small molecule, a biotherapeutic, a biologic, a pharmaceutical, and combinations thereof.

6. The non-invasive, in situ forming depot for delivery of a therapeutic agent of claim 1, wherein the one or more heterodimerizing, synthetic leucine zippers are attached to a surface of the therapeutic agent, optionally attached via crosslinking, physical attachment or genetic fusion, optionally a heterobifunctional crosslinker.

7. (canceled)

8. (canceled)

9. The non-invasive, in situ forming depot for delivery of a therapeutic agent of claim 1, wherein the therapeutic agent comprises a mesenchymal stem cell with a natural ability to migrate to sites of inflammation in vivo.

10. The non-invasive, in situ forming depot for delivery of a therapeutic agent of claim 1, wherein the target site is an infarct site, wherein the therapeutic agent comprises a stem cell.

11. (canceled)

12. The non-invasive, in situ forming depot for delivery of a therapeutic agent of claim 1, wherein the non-invasive, in situ forming depots are configured to be sequentially administered to a subject, whereby one or more layers of the in situ forming depots can be formed at a target site, optionally wherein the one or more heterodimerizing, synthetic leucine zippers cause physical multivalent crosslinking, wherein physical multivalent crosslinking causes retention and accumulation of the therapeutic agent at the target site and/or wherein each layer serves as an additional capturing surface for a subsequent dose of a non-invasive, in situ forming depot.

13. (canceled)

14. (canceled)

15. The non-invasive, in situ forming depot for delivery of a therapeutic agent of claim 1, wherein the non-invasive, in situ forming depots are configured to be sequentially administered to a subject, whereby each sequential administration is configured to provide a different therapeutic agent and/or a different dosage of the therapeutic agent.

16. (canceled)

17. The non-invasive, in situ forming depot for delivery of a therapeutic agent of claim 1, wherein the non-invasive, in situ forming depots are configured for intravenous administration subcutaneous, intramuscular, local, intracranial and/or intraarterial administration.

18. The non-invasive, in situ forming depot for delivery of a therapeutic agent of claim 1, wherein multivalent crosslinking of the one or more leucine zippers increases resistance to venous washout, allowing the therapeutic agent to persist at the target site long term.

19. (canceled)

20. The non-invasive, in situ forming depot for delivery of a therapeutic agent of claim 1, wherein the non-invasive, in situ forming depots, including the therapeutic agent, are configured to deposit at the target site at a concentration of at least about 2 times, about 5 times, about 10 times, about 20 times, about 50 times, or more, as compared to a therapeutic agent without the non-invasive, in situ forming depots.

21. The non-invasive, in situ forming depot for delivery of a therapeutic agent of claim 1, wherein the one or more leucine zippers comprise an amino acid or nucleotide sequence of any of SEQ ID NOs. 1-8, or a variant thereof substantially identical to any of SEQ ID NOs. 1-8, optionally wherein the variant has a homology of at least about 75%, 80%, 85%, 90%, 95%, 99% or more as compared to any of SEQ ID NOs. 1-8.

22. (canceled)

23. (canceled)

24. (canceled)

25. The non-invasive, in situ forming depot for delivery of a therapeutic agent of claim 1, wherein the non-invasive, in situ forming depots are configured to be disassembled via competition-mediated disassembly.

26. A method of treating, ameliorating and/or preventing a condition in a subject, the method comprising:
providing a subject in need of treatment, amelioration and/or prevention of a condition; and
administering to the subject a non-invasive, in situ forming depot of claim 1.

27. The method of claim 26, wherein the subject is a human subject, optionally wherein the subject is suffering from an inflammatory condition, optionally wherein the subject is suffering from myocardial infarction, ischemia, cancer, arthritis, joint disease, and/or limb ischemia.

28. The method of claim 26, wherein the subject is suffering from myocardial infarction or is susceptible to suffering from myocardial infarction, optionally wherein the subject administered the non-invasive, in situ forming depot shows improved cardiac function, defined as higher fractional shortening (FS), optionally about 10% to about 130% higher FS; decreased systolic LV diameter, optionally about 10% to about 40% lower systolic LV diameter; improved ejection fraction, optionally about 10% to about 100% higher ejection fraction; and/or less fibrosis, optionally about 15% to about 60% lower fibrosis.

29. (canceled)

30. (canceled)

31. (canceled)

32. A leucine zipper comprising an amino acid sequence of any of SEQ ID NOs. 1, 3, 5 or 7, or a substantially identical variant thereof, optionally wherein the variant has a homology of at least about 75%, 80%, 85%, 90%, 95%, 99% or more as compared to any of SEQ ID NOs. 1, 3, 5 or 7, optionally wherein amino acids 22-47 of SEQ ID NOs. 1, 3, 5 or 7 are substantially conserved.

33. (canceled)

34. (canceled)

35. (canceled)

36. (canceled)

37. A leucine zipper comprising nucleotide sequence of any of SEQ ID NOs. 2, 4, 6 or 8, or a substantially identical variant thereof, optionally wherein the variant has a homology of at least about 75%, 80%, 85%, 90%, 95%, 99% or more as compared to any of SEQ ID NOs. 2, 4, 6 or 8.

38. (canceled)

39. (canceled)

40. (canceled)

41. (canceled)

42. (canceled)

43. A pharmaceutical composition comprising a leucine zipper-based therapeutic in combination with a stem cell therapeutic, optionally wherein the stem cell therapeutic comprises an extracellular vesicle (EV), wherein one or more stem cells in the stem cell therapeutic are tagged with a leucine zipper, optionally wherein the leucine zipper comprises a leucine zipper of claim 32.

44. (canceled)

45. (canceled)

46. (canceled)

47. (canceled)


SEQUENCE LISTING (12 sequences)

SEQ ID NO: 1 - protein, 47 aa, synthetic construct
  REGION 1..47: note = Artificially synthesized amino acide sequence
NLVAQLENEVASLENENETLKKKILHKKDLIAYLEKEIANLRKKIEE

SEQ ID NO: 2 - DNA, 141 bp, synthetic construct
  misc_feature 1..141: note = Artificially syntheszied nucleotide sequence
  source 1..141: mol_type = other DNA
aacttagttgctcaactagagaatgaagtagcgagcctggaaaacgagaacgaaaccctgaaaaagaagatcttgcacaaaaaggacctgattgcatacttggagaaagagatcgctaatctgcgtaaaaagatcgaggaa

SEQ ID NO: 3 - protein, 47 aa, synthetic construct
  REGION 1..47: note = Artificially synthesized amino acid sequence
NLVAQLENEVASLENENETLKNKNLQKKNLIAYLEQEIANLRKKIEG

SEQ ID NO: 4 - DNA, 141 bp, synthetic construct
  misc_feature 1..141: note = Artificially synthesized nucleotide sequence
  source 1..141: mol_type = other DNA
aaccttgtggcccaattagagaacgaggtggcctccctggaaaatgagaacgaaacattaaaaaacaaaaacttgcagaagaaaaacttaatcgcctacttggaacaagagattgctaacttacgcaagaagattgaaggg

SEQ ID NO: 5 - protein, 47 aa, synthetic construct
  REGION 1..47: note = Artificially synthesized amino acid sequence
NLVAQLENEVASLENENETLKKKNLHLKDLIAYLEKEIANLRKKIEE

SEQ ID NO: 6 - DNA, 141 bp, synthetic construct
  misc_feature 1..141: note = Artificially synthesized nucleotide sequence
  source 1..141: mol_type = other DNA
aacttagttgctcaactagagaatgaagtagcgagcctggaaaacgagaacgaaaccctgaaaaagaagaacttgcacctaaaggacctgattgcatacttggagaaagagatcgctaatctgcgtaaaaagatcgaggaa

SEQ ID NO: 7 - protein, 47 aa, synthetic construct
  REGION 1..47: note = Artificially synthesized amino acid sequence
NLVAQLENEVASLENENETLKNKNLQKKNLIAYLEQEIANLRNKIEG

SEQ ID NO: 8 - DNA, 141 bp, synthetic construct
  misc_feature 1..141: note = Artificially synthesized nucleotide sequence
  source 1..141: mol_type = other DNA
aaccttgtggcccaattagagaacgaggtggcctccctggaaaatgagaacgaaacattaaaaaacaaaaacttgcagaagaaaaacttaatcgcctacttggaacaagagattgctaacttacgcaacaagattgaaggg

SEQ ID NO: 9 - protein, 49 aa, synthetic construct
  REGION 1..49: note = Artificially synthesized amino acid sequence
ARNAYLRKKIARLKKDNLQLERDEQNLEKIIANLRDEIARLEEVASHEQ

SEQ ID NO: 10 - protein, 44 aa, synthetic construct
  REGION 1..44: note = Artificially synthesized amino acid sequence
NELESLENKKEELKNRNEELKQKREQLKQKLAALRNKLDAYKRL

SEQ ID NO: 11 - protein, 40 aa, synthetic construct
  REGION 1..40: note = Artificially synthesized amino acid sequence
ELTDELKNKKEALRKDAALLELASLENEIANLEKEIAYFK

SEQ ID NO: 12 - protein, 41 aa, synthetic construct
  REGION 1..41: note = Artificially synthesized amino acid sequence
LTEECLEKLEKELFSARTTARENGETIREYAKYARVVIIGP